(19) United States
(12) Patent Application Publication
(10) Pub. No.: US 2008/0287517 A1
(43) Pub. Date: Nov. 20, 2008

(54) Pyrazole Derivatives as Cb1 Modulators

(76) Inventors: Leifeng Cheng, Molndal (SE); Maria Jonforsen, Molndal (SE); Peter Schell, Molndal (SE)

Correspondence Address: MORGAN LEWIS & BOCKIUS LLP, 1111 PENNSYLVANIA AVENUE NW, WASHINGTON, DC, 20004, US

(21) Appl. No.: 11/996,115

(22) PCT Filed: Jul. 17, 2006
(86) PCT No.: PCT/GB2006/002631

§ 371 Date: Aug. 4, 2008

(30) Foreign Application Priority Data
Jul. 19, 2005 (GB) 0514738.4

(51) Int. Cl. A61K 31/415; C07D 231/14; A61P 9/00; A61P 3/00; A61P 25/00; A61P 37/00

(52) U.S. Cl. 514/406; 548/374.1

(57) ABSTRACT

The present invention relates to compounds of formula I

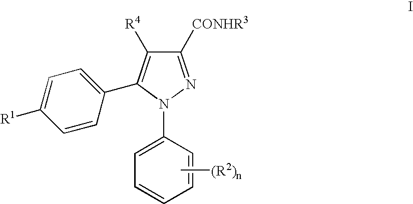

wherein R¹ represents a group R⁵O— in which R⁵ represents a C₃₋₇alkyl group substituted by one or more fluoro or R⁵ represents a C₃₋₇alkylsulphonyl group which is optionally substituted by one or more fluoro; R² represents a C₁₋₄alkyl group, hydroxy, fluoro, chloro or cyano wherein each R² is independently selected when n is >1; R³ represents a) cyclohexyl optionally substituted by one or more of the following: hydroxy, fluoro, amino, mono or diC₁₋₃alkylamino, carboxy or a C₁₋₄alkoxycarbonyl group b) piperidino substituted by one or more hydroxy c) unsubstituted piperidino but only when one of the following applies: R⁴ represents cyano or R¹ represents 3-fluoropropylsulphonyloxy or R¹ represents 3,3,3-trifluoropropoxy or R¹ represents 3-fluoropropoxy or R² is methyl d) phenyl substituted by one or more of the following: hydroxy, halo or a C₁₋₄alkyl group e) pyridyl substituted by a C₁₋₄alkyl group or f) a C₄₋₉alkyl group; R⁴ represents cyano or methyl; and n is 1, 2 or 3 and pharmaceutically acceptable salts thereof and processes for preparing such compounds, their use in the treatment of obesity, psychiatric and neurological disorders, to methods for their therapeutic use and to pharmaceutical compositions containing them.

### FIELD OF INVENTION

The present invention relates to certain compounds of formula I, to processes for preparing such compounds, to their use in the treatment of obesity, psychiatric and neurological disorders, to methods for their therapeutic use and to pharmaceutical compositions containing them.

### BACKGROUND OF THE INVENTION

It is known that certain CB₁ modulators (known as antagonists or inverse agonists) are useful in the treatment of obesity, psychiatric and neurological disorders (WO01/70700 EP 658,546 and EP 656,354).

Pyrazoles having anti-inflammatory activity are disclosed in WO 95/15316, WO96/38418, WO97/11704, WO99/64415, EP 418 845 and WO2004050632. WO2004050632 discloses 1,1-dimethylethyl [2-[4-[3-[(ethylmethylamino)carbonyl]-1-(4-methoxyphenyl)-1H-pyrazol-5-yl]phenoxy]ethyl]carbamate, 5-[4-(2-aminoethoxy)phenyl]-N-ethyl-1-(4-methoxyphenyl)-N-methyl-1H-pyrazole-3-carboxamide, 1-[[5-[4-(2-aminoethoxy)phenyl]-1-(4-methoxyphenyl)-1H-pyrazol-3-yl]carbonyl]piperidine and 1,1-dimethylethyl [2-[4-[1-(4-methoxyphenyl)-3-(1-piperidinylcarbonyl)-1H-pyrazol-5-yl]phenoxy]ethyl]-carbamate. All compounds exemplified in WO2004050632 and salts thereof are excluded from the scope of the compound claims of the present invention.

1,5-Diarylpyrazole-3-carboxamide derivatives are disclosed as having CB₁ modulatory activity in U.S. Pat. No. 5,624,941, WO01/29007, WO2004/052864, WO03/020217, US 2004/0119972, Journal of Medicinal Chemistry, 46(4), 642-645 2003, Bioorganic & Medicinal Chemistry Letters, 14(10), 2393-2396 2004, Biochemical Pharmacology, 60(9), 1315-1323 2000, Journal of Medicinal Chemistry, 42(4), 769-776 1999 and U.S. Pat. Appl. Publ. US 2003199536. All compounds disclosed in these documents are disclaimed from the present application.

Co-pending application number PCT/GB2005/000534 discloses CB1 antagonists of formula (A)

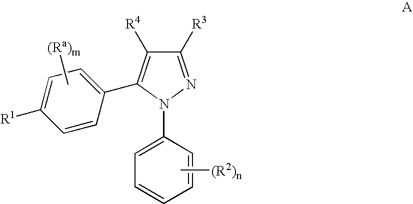

and pharmaceutically acceptable salts thereof, in which
R¹ represents a) a C₁₋₃alkoxy group substituted by one or more of the following i) fluoro ii) a group NR^{c}R^{d} in which R^{c} and R^{d} independently represent H, a C₁₋₆alkyl group or C₁₋₆alkoxycarbonyl group provided that one of R^{c} and R^{d} is other than H or iii) a 1,3-dioxolan-2-yl group b) R¹ represents a C₄₋₆alkoxy group optionally substituted by one or more of the following i) fluoro ii) a group NR^{c}R^{d} in which R^{c} and R^{d} independently represent H, a C₁₋₆alkyl group or C₁₋₆alkoxycarbonyl group provided that one of R^{c} and R^{d} is other than H or iii) a 1,3-dioxolan-2-yl group c) a group of formula phenyl(CH₂)ₚO— in which p is 1, 2 or 3 and the phenyl ring is optionally substituted by 1, 2 or 3 groups represented by Z, d) a group R⁵S(O)₂O or R⁵S(O)₂NH in which R⁵ represents a C₁₋₆alkyl group optionally substituted by one or more fluoro, or R⁵ represents phenyl or a heteroaryl group each of which is optionally substituted by 1, 2 or 3 groups represented by Z e) a group of formula (R⁶)₃Si in which R¹ represents a C₁₋₆alkyl group which may be the same or different or f) a group of formula R^{b}O(CO)O in which R^{b} represents a C₁₋₆alkyl group optionally substituted by one or more fluoro;
R^{a} represents halo, a C₁₋₃alkyl group or a C₁₋₃alkoxy group;
m is 0, 1, 2 or 3;
R² represents a C₁₋₃alkyl group, a C₁₋₃alkoxy group, hydroxy, nitro, cyano or halo
n is 0, 1, 2 or 3;
R³ represents
a) a group X—Y—NR⁷R⁸ in which X is CO or SO₂,
Y is absent or represents NH optionally substituted by a C₁₋₃alkyl group;
and R⁷ and R⁸ independently represent:
a C₁₋₆alkyl group optionally substituted by 1, 2, or 3 groups represented by W;
a C₃₋₁₅cycloalkyl group optionally substituted by 1, 2, or 3 groups represented by W;
a (C₃₋₁₅cycloalkyl)C₁₋₃alkylene group optionally substituted by 1, 2, or 3 groups represented by W;
a group —(CH₂)ᵣ(phenyl)ₛ in which r is 0, 1, 2, 3 or 4, s is 1 when r is 0 otherwise s is 1 or 2 and the phenyl groups are optionally independently substituted by one, two or three groups represented by Z;
a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups, hydroxy or benzyl;
a group —(CH₂)ₜHet in which t is 0, 1, 2, 3 or 4, and the alkylene chain is optionally substituted by one or more C₁₋₃alkyl groups and Het represents an aromatic heterocycle optionally substituted by one, two or three groups selected from a C₁₋₅alkyl group, a C₁₋₅alkoxy group or halo wherein the alkyl and alkoxy group are optionally independently substituted by one of more fluoro;
or R⁷ represents H and R⁸ is as defined above;
or R⁷ and R⁸ together with the nitrogen atom to which they are attached represent a saturated or partially unsaturated 5 to 8 membered heterocyclic group containing one nitrogen and optionally one of the following: oxygen, sulphur or an additional nitrogen;
wherein the heterocyclic group is optionally substituted by one or more C₁₋₃alkyl groups, hydroxy, fluoro or benzyl;
or b) oxazolyl, isoxazolyl, thiazolyl, isothiazolyl, oxadiazolyl, thiadiazolyl, pyrrolyl, pyrazolyl, imidazolyl, triazolyl, tetrazolyl, thienyl, furyl or oxazolinyl, each optionally substituted by 1, 2 or 3 groups Z;
R⁴ represents H, halo, hydroxy, cyano, a C₁₋₆alkyl group, a C₁₋₆alkoxy group or a C₁₋₆alkoxyC₁₋₆alkylene group which contains a maximum of 6 carbon atoms, each of which groups is optionally substituted by one or more fluoro or cyano;
Z represents a C₁₋₃alkyl group, a C₁₋₃alkoxy group, hydroxy, halo, trifluoromethyl, trifluoromethylthio, difluoromethoxy, trifluoromethoxy, trifluoromethylsulphonyl, nitro, amino, mono or di C₁₋₃alkylamino, C₁₋₃alkylsulphonyl, C₁₋₃alkoxycarbonyl, carboxy, cyano, carbamoyl, mono or di C₁₋₃alkyl carbamoyl and acetyl; and
W represents hydroxy, fluoro, a C₁₋₃alkyl group, a C₁₋₃alkoxy group, amino, mono or di C₁₋₃alkylamino, a C₁₋₆alkoxycarbonyl group or a heterocyclic amine selected from morpholinyl, pyrrolidinyl, piperidinyl or piperazinyl in which the heterocyclic amine is optionally substituted by a C₁₋₃alkyl group or hydroxyl;
but excluding 1,1-dimethylethyl [2-[4-[3-[(ethylmethylamino)carbonyl]-1-(4-methoxyphenyl)-1H-pyrazol-5-yl]phenoxy]ethyl]carbamate and 1,1-dimethylethyl [2-[4-[1-(4-methoxyphenyl)-3-(1-piperidinylcarbonyl)-1H-pyrazol-5-yl]phenoxy]ethyl]carbamate. Compounds exemplified in this application are disclaimed from the present application.

However, there is still a need for CB₁ modulators with improved physicochemical properties and/or DMPK properties and/or pharmacodynamic properties. A select group of compounds has been found that addresses this need.

### DESCRIPTION OF THE INVENTION

The invention relates to a compound of formula (I)

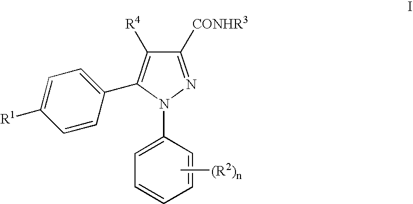

R¹ represents a group R⁵O— in which R⁵ represents a C₃₋₇alkyl group substituted by one or more fluoro or R⁵ represents a C₃₋₇alkylsulphonyl group which is optionally substituted by one or more fluoro;
R² represents a C₁₋₄alkyl group, hydroxy, fluoro, chloro or cyano wherein each R² is independently selected when n is >1;
R³ represents a) cyclohexyl optionally substituted by one or more of the following: hydroxy, fluoro, amino, mono or diC₁₋₃alkylamino, carboxy or a C₁₋₄alkoxycarbonyl group
b) piperidino substituted by one or more hydroxy c) unsubstituted piperidino but only when one of the following applies: R⁴ represents cyano or R¹ represents 3-fluoropropylsulphonyloxy or R¹ represents 3,3,3-trifluoropropoxy or R¹ represents 3-fluoropropoxy or R¹ is methyl d) phenyl substituted by one or more of the following: hydroxy, halo or a C₁₋₄alkyl group e) pyridyl substituted by a C₁₋₄alkyl group or f) a C₄₋₉alkyl group;
R⁴ represents cyano or methyl; and
n is 1, 2 or 3
and pharmaceutically acceptable salts thereof.

It will be understood that when n is 2 or 3 then the groups R² are independently selected so that they may be the same or different.

In a first group of compounds of formula I, R¹ represents n-butylsulfonyloxy, n-propylsulfonyloxy, 3-methylbutylsulfonyloxy, 4,4,4-trifluorobutyl-1-sulfonyloxy, 4-fluorobutyl-1-sulfonyloxy, 3,3,3-trifluoropropyl-1-sulfonyloxy, 3-fluoropropyl-1-sulfonyloxy, 4,4,4-trifluorobutoxy, 4-fluorobutoxy, 3,3,3-trifluoropropoxy or 3-fluoropropoxy.

In a second group of compounds of formula I, R² represents chloro, fluoro, cyano, hydroxy or methyl and n is 1, 2 or 3.

In a third group of compounds of formula I, R³ represents cyclohexyl substituted by one or more of the following: hydroxy, fluoro, amino, mono or di C₁₋₃alkylamino, carboxy or a C₁₋₄alkoxycarbonyl group; for example 2-hydroxycyclohexyl, 3-hydroxycyclohexyl, 4-hydroxycyclohexyl, 2-aminocyclohexyl, 3-aminocyclohexyl, 2-dimethylaminocyclohexyl, 3-dimethylaminocyclohexyl or 4,4-difluorocyclohexyl. In one group of compounds the substituent is in the 2 or 3 position. In another group of compounds the substituent on the cyclohexyl ring is in the cis conformation with respect to the nitrogen of the amide. In another group of compounds the substituent on the cyclohexyl ring is in the trans conformation with respect to the nitrogen of the amide.

In a fourth group of compounds of formula I, R³ represents piperidino substituted by one or more hydroxy for example 3-hydroxypiperidino or 4 hydroxypiperidino.

In a fifth group of compounds of formula I, R³ represents unsubstituted piperidino but only when one of the following applies: R⁴ represents cyano or R³ represents 3-fluoropropylsulphonyloxy or R¹ represents 3,3,3-trifluoropropoxy or R¹ represents 3-fluoropropoxy or R² is methyl.

In a sixth group of compounds of formula I, R³ represents phenyl substituted by one or more of the following: hydroxy, halo or a C₁₋₄alkyl group, for example 3,4-difluoro-2-hydroxyphenyl.

In a seventh group of compounds of formula I, R³ represents pyridyl substituted by a C₁₋₄alkyl group or fluoro, for example methylpyridyl e.g. 5-methyl-2-pyridyl or for example fluoro pyridyl e.g. 5-fluoro-2-pyridyl.

In an eighth group of compounds of formula I, R³ represents a C₄₋₉alkyl group for example 2-ethyl-1-butyl.

In a ninth group of compounds of formula I, R⁴ represents cyano.

In a tenth group of compounds of formula I, R⁴ represents methyl.

Particularly the fluoro substitution in R⁵ is in the terminal carbon atom of the R¹ chain. “Pharmaceutically acceptable salt”, where such salts are possible, includes both pharmaceutically acceptable acid and base addition salts. A suitable pharmaceutically acceptable salt of a compound of Formula I is, for example, an acid-addition salt of a compound of Formula I which is sufficiently basic, for example an acid-addition salt with an inorganic or organic acid such as hydrochloric, hydrobromic, sulphuric, trifluoroacetic, citric or maleic acid; or, for example a salt of a compound of Formula I which is sufficiently acidic, for example an alkali or alkaline earth metal salt such as a sodium, calcium or magnesium salt, or an ammonium salt, or a salt with an organic base such as methylamine, dimethylamine, trimethylamine, piperidine, morpholine or tris-(2-hydroxyethyl)amine.

Throughout the specification and the appended claims, a given chemical formula or name shall encompass all stereo and optical isomers and racemates thereof as well as mixtures in different proportions of the separate enantiomers, where such isomers and enantiomers exist, as well as pharmaceutically acceptable salts thereof and solvates thereof such as for instance hydrates. Isomers may be separated using conventional techniques, e.g. chromatography or fractional crystallisation. The enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions which will not cause racemisation or epimerisation, or by derivatisation, with a chiral reagent. All stereoisomers are included within the scope of the invention. All tautomers, where possible, are included within the scope of the invention. The present invention also encompasses compounds containing one or more isotopes for example ¹⁴C, ¹¹C or ¹⁹F and their use as isotopically labelled compounds for pharmacological and metabolic studies.

The present invention also encompasses prodrugs of a compound of formula I that is compounds which are converted into a compound of formula I in vivo.

The following definitions shall apply throughout the specification and the appended claims.

Unless otherwise stated or indicated, the term “alkyl” denotes either a straight or branched iso-butyl, sec-butyl and t-butyl. Preferred alkyl groups are methyl, ethyl, propyl, isopropyl and tertiary butyl.

Unless otherwise stated or indicated, the term “alkoxy” denotes a group O-alkyl, wherein alkyl is as defined above.

Unless otherwise stated or indicated, the term “halogen” shall mean fluorine, chlorine, bromine or iodine.

Specific compounds of the invention are one or more of the following:
4-{4-cyano-1-(2,4-dichlorophenyl)-3-[(piperidin-1-ylamino)carbonyl]-1H-pyrazol-5-yl}phenyl 3,3,3-trifluoropropane-1-sulfonate;
4-{4-cyano-1-(2,4-dichlorophenyl)-3-[(piperidin-1-ylamino)carbonyl]-1H-pyrazol-5-yl}phenyl 3-methylbutane-1-sulfonate;
4-[1-(2,4-dichlorophenyl)-3-({[(1R,2S)-2-hydroxycyclohexyl]amino}carbonyl)-4-methyl-1H-pyrazol-5-yl]phenyl 3,3,3-trifluoropropane-1-sulfonate;
4-[1-(2,4-dichlorophenyl)-3-({[(1S,2R)-2-hydroxycyclohexyl]amino}carbonyl)-4-methyl-1H-pyrazol-5-yl]phenyl 3,3,3-trifluoropropane-1-sulfonate;
4-(1-(2,4-dichlorophenyl)-3-{[(5-fluoropyridin-2-yl)amino]carbonyl}-4-methyl-1H-pyrazol-5-yl)phenyl 3,3,3-trifluoropropane-1-sulfonate;
4-(1-(2,4-dichlorophenyl)-3-{[(3,4-difluoro-2-hydroxyphenyl)amino]carbonyl}-4-methyl-1H-pyrazol-5-yl)phenyl 3,3,3-trifluoropropane-1-sulfonate;
4-{1-(2,4-dichlorophenyl)-4-methyl-3-[(piperidin-1-ylamino)carbonyl]-1H-pyrazol-5-yl}phenyl 3-fluoropropane-1-sulfonate;
3,3,3-trifluoropropane-1-sulfonic acid 4-[2-(2,4-dichlorophenyl)-5-(4-hydroxy-piperidin-1-ylcarbamoyl)-4-methyl-2H-pyrazol-3-yl]phenyl ester;
3,3,3-trifluoropropane-1-sulfonic acid 4-[2-(2,4-dichlorophenyl)-5-(3-hydroxy-piperidin-1-ylcarbamoyl)-4-methyl-2H-pyrazol-3-yl]phenyl ester;
3-methylbutane-1-sulfonic acid 4-[2-(2,4-dichlorophenyl)-4-methyl-5-(5-methyl-pyridin-2-ylcarbamoyl)-2H-pyrazol-3-yl]phenyl ester;
3,3,3-trifluoropropane-1-sulfonic acid 4-[2-(2,4-dichlorophenyl)-4-methyl-5-(5-methyl-pyridin-2-ylcarbamoyl)-2H-pyrazol-3-yl]phenyl ester;
(−)-4-[1-(2,4-dichlorophenyl)-3-({[cis-2-hydroxycyclohexyl]amino}carbonyl)-4-methyl-1H-pyrazol-5-yl]phenyl 3,3,3-trifluoropropane-1-sulfonate;
(+)-4-[1-(2,4-dichlorophenyl)-3-({[cis-2-hydroxycyclohexyl]amino}carbonyl)-4-methyl-1H-pyrazol-5-yl]phenyl 3,3,3-trifluoropropane-1-sulfonate;
4-[1-(2,4-dichlorophenyl)-3-({[3-(dimethylamino)cyclohexyl]amino}carbonyl)-4-methyl-1H-pyrazol-5-yl]phenyl 3,3,3-trifluoropropane-1-sulfonate;
4-[1-(2,4-dichlorophenyl)-3-({[trans-3-(dimethylamino)cyclohexyl]amino}carbonyl)-4-methyl-1H-pyrazol-5-yl]phenyl 3,3,3-trifluoropropane-1-sulfonate;
4-[1-(2,4-dichlorophenyl)-3-({[cis-3-(dimethylamino)cyclohexyl]amino}carbonyl)-4-methyl-1H-pyrazol-5-yl]phenyl 3,3,3-trifluoropropane-1-sulfonate;
4-[3-({[cis-3-aminocyclohexyl]amino}carbonyl)-1-(2,4-dichlorophenyl)-4-methyl-1H-pyrazol-5-yl]phenyl 3,3,3-trifluoropropane-1-sulfonate;
4-[3-[({trans-3-[(tert-butoxycarbonyl)amino]cyclohexyl}amino)carbonyl]-1-(2,4-dichlorophenyl)-4-methyl-1H-pyrazol-5-yl]phenyl 3,3,3-trifluoropropane-1-sulfonate;
1-(2,4-dichlorophenyl)-4-methyl-N-piperidin-1-yl-5-[4-(3,3,3-trifluoropropoxy)phenyl]-1H-pyrazole-3-carboxamide;
N-cyclohexyl-1-(2,4-dichlorophenyl)-4-methyl-5-[4-(3,3,3-trifluoropropoxy)phenyl]-1H-pyrazole-3-carboxamide;
1-(2,4-dichlorophenyl)-N-[(cis)-2-hydroxycyclohexyl]-4-methyl-5-[4-(3,3,3-trifluoropropoxy)phenyl]-1H-pyrazole-3-carboxamide;
1-(2,4-dichlorophenyl)-N-(4,4-difluorocyclohexyl)-4-methyl-5-[4-(3,3,3-trifluoropropoxy)phenyl]-1H-pyrazole-3-carboxamide;
1-(2,4-dichlorophenyl)-4-methyl-N-(5-methylpyridin-2-yl)-5-[4-(3,3,3-trifluoropropoxy)phenyl]-1H-pyrazole-3-carboxamide;
4-[1-(2-chlorophenyl)-3-{[(1S,2R)-2-hydroxycyclohexyl]carbamoyl}-4-methyl-1H-pyrazol-5-yl]phenyl 3,3,3-trifluoropropane-1-sulfonate;
4-[1-(2-chlorophenyl)-3-{[(1R,2S)-2-hydroxycyclohexyl]carbamoyl}-4-methyl-1H-pyrazol-5-yl]phenyl 3,3,3-trifluoropropane-1-sulfonate:
4-[1-(2-chlorophenyl)-3-(cyclohexylcarbamoyl)-4-methyl-1H-pyrazol-5-yl]phenyl 3,3,3-trifluoropropane-1-sulfonate;
4-{1-(4-chloro-2-methylphenyl)-4-methyl-3-[(piperidin-1-ylamino)carbonyl]-1H-pyrazol-5-yl}phenyl 3,3,3-trifluoropropane-1-sulfonate;
4-[1-(4-chloro-2-methylphenyl)-3-({[(1S,2R)-2-hydroxycyclohexyl]amino}carbonyl)-4-methyl-1H-pyrazol-5-yl]phenyl 3,3,3-trifluoropropane-1-sulfonate;
4-[1-(4-chloro-2-methylphenyl)-3-({[(1R,2S)-2-hydroxycyclohexyl]amino}carbonyl)-4-methyl-1H-pyrazol-5-yl]phenyl 3,3,3-trifluoropropane-1-sulfonate;
4-[1-(2,4-dichlorophenyl)-3-({[(1S,3R)-3-hydroxycyclohexyl]amino}carbonyl)-4-methyl-1H-pyrazol-5-yl]phenyl 3,3,3-trifluoropropane-1-sulfonate;
4-[1-(2,4-dichlorophenyl)-3-({[(1R,3S)-3-hydroxycyclohexyl]amino}carbonyl)-4-methyl-1H-pyrazol-5-yl]phenyl 3,3,3-trifluoropropane-1-sulfonate;
4-[1-(2,4-dichlorophenyl)-3-({[(1S,3S)-3-hydroxycyclohexyl]amino}carbonyl)-4-methyl-1H-pyrazol-5-yl]phenyl 3,3,3-trifluoropropane-1-sulfonate;
4-[1-(2,4-dichlorophenyl)-3-({[(1R,3R)-3-hydroxycyclohexyl]amino}carbonyl)-4-methyl-1H-pyrazol-5-yl]phenyl 3,3,3-trifluoropropane-1-sulfonate;
1-(2,4-dichlorophenyl)-5-[4-(3-fluoropropoxy)phenyl]-4-methyl-N-piperidin-1-yl-1H-pyrazole-3-carboxamide;
1-(2,4-dichlorophenyl)-5-[4-(3-fluoropropoxy)phenyl]-N-[(cis)-2-hydroxycyclohexyl]-4-methyl-1H-pyrazole-3-carboxamide;
1-(2,4-dichlorophenyl)-N-(4,4-difluorocyclohexyl)-5-[4-(3-fluoropropoxy)phenyl]-4-methyl-1H-pyrazole-3-carboxamide;
1-(2,4-dichlorophenyl)-5-[4-(3-fluoropropoxy)phenyl]-4-methyl-N-(5-methylpyridin-2-yl)-1H-pyrazole-3-carboxamide;
1-(2,4-Dichlorophenyl)-4-methyl-5-[4-(3,3,3-trifluoropropoxy)-phenyl]-1H-pyrazole-3-carboxylic acid (2-hydroxycyclohexyl)amide
1-(2,4-dichlorophenyl)-4-methyl-5-[4-(3,3,3-trifluoropropoxy)phenyl]-1H-pyrazole-3-carboxylic acid (3-hydroxycyclohexyl)amide;
3-fluoropropane-1-sulfonic acid 4-[2-(2,4-dichlorophenyl)-5-((1S,2R)-2-hydroxycyclohexylcarbamoyl)-4-methyl-2H-pyrazol-3-yl]phenyl ester;
4,4,4-trifluorobutane-1-sulfonic acid 4-[2-(3-cyano-5-fluorophenyl)-5-(1-ethylbutylcarbamoyl)-4-methyl-2H-pyrazol-3-yl]phenyl ester;
3,3,3-trifluoropropane-1-sulfonic acid 4-[2-(3-cyano-5-fluorophenyl)-5-(4,4-difluoro-cyclohexylcarbamoyl)-4-methyl-2H-pyrazol-3-yl]phenyl ester;
3,3,3-trifluoropropane-1-sulfonic acid 4-[2-(3-cyanophenyl)-5-(4,4-difluoro-cyclohexylcarbamoyl)-4-methyl-2H-pyrazol-3-yl]phenyl ester;
3,3,3-trifluoropropane-1-sulfonic acid 4-[5-(2-aminocyclohexylcarbamoyl)-2-(3-cyano-5-fluorophenyl)-4-methyl-2H-pyrazol-3-yl]-phenyl ester;
3,3,3-trifluoropropane-1-sulfonic acid 4-[2-(3-cyano-5-fluorophenyl) -5-(3-dimethylaminocyclohexylcarbamoyl)-4-methyl-2H-pyrazol-3-yl]phenyl ester;
3,3,3-trifluoropropane-1-sulfonic acid 4-[2-(3-cyano-5-fluorophenyl)-5-((1S,2R)-2-hydroxycyclohexylcarbamoyl)-4-methyl-2H-pyrazol-3-yl]phenyl ester;
3,3,3-trifluoropropane-1-sulfonic acid 4-[2-(3-cyanophenyl)-5-(2-hydroxy-cyclohexylcarbamoyl)-4-methyl-2H-pyrazol-3-yl]phenyl ester;
3,3,3-trifluoropropane-1-sulfonic acid 4-[2-(3-cyano-5-fluorophenyl)-5-(3-hydroxy-cyclohexylcarbamoyl)-4-methyl-2H-pyrazol-3-yl]phenyl ester;
N-cyclohexyl-1-(2,4-dichlorophenyl)-5-[4-(3-fluoropropoxy)phenyl]-4-methyl-1H-pyrazole-3-carboxamide;
3,3,3-trifluoropropane-1-sulfonic acid 4-[2-(2-chlorophenyl)-5-(2-hydroxy-cyclohexylcarbamoyl)-4-methyl-2H-pyrazol-3-yl]phenyl ester; and
3,3,3-trifluoropropane-1-sulfonic acid 4-[2-(2-chlorophenyl)-5-(4,4-difluorocyclohexylcarbamoyl)-4-methyl-2H-pyrazol-3-yl]phenyl ester;
   as well as pharmaceutically acceptable salts thereof.

### Methods of Preparation

The compounds of the invention may be prepared as outlined below according to any of the following methods. However, the invention is not limited to these methods, the compounds may also be prepared as described for structurally related compounds in the prior art.

Compounds of formula I in which R¹ represents a) a C₃₋₆alkoxy group substituted by one or more fluoro or b) a group of formula phenyl(CH₂)ₚO— in which p is 1, 2 or 3 and the phenyl ring is optionally substituted by 1, 2 or 3 groups represented by Z, or c) a group R⁵S(O)₂O may be prepared by reacting a compound of formula II

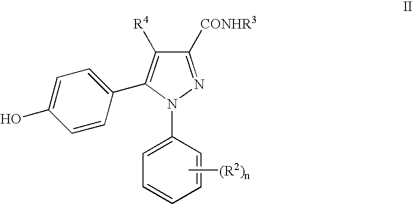

in which R², R³, R⁴, and n are as previously defined with a group R^{1A}-X in which R^{1A} represents a group such that R^{1A}O represents R¹ and X represents a leaving group for example halo, at a temperature in the range of −25 to 150° C., in the presence of an inert solvent, for example dichloromethane, and optionally in the presence of a base for example triethylamine or pyridine.

Compounds of formula I in which R¹, R², R³, R⁴, and n are as previously defined may be prepared by reacting a compound of formula III

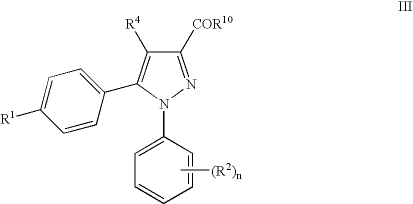

in which R¹, R², R⁴ and n are as previously defined and R¹⁰ represents OH or a C₁₋₆alkoxy group or chloro with a compound of formula IV or a salt thereof

R³NH₂ IV

in which R¹ is as previously defined, for example in an inert solvent, for example toluene, in the presence of a Lewis Acid, for example triethylaluminium, at a temperature in the range of −25° C. to 150° C. when R¹⁰ is a C₁₋₆alkoxy group; or alternatively when R¹⁰ is OH by reacting a compound of formula III with a chlorinating agent for example oxalyl chloride, and then reacting the acid chloride produced with an amine of formula IV in an inert solvent, for example dichloromethane, in the presence of a base, for example triethylamine or pyridine, at a temperature in the range of −25° C. to 150° C.

Certain intermediate compounds of Formula II are believed to be novel and form part of the present invention. Compounds of formula II may be prepared as described in the Examples.

It will be appreciated by those skilled in the art that during the reaction sequence certain functional groups will require protection followed by deprotection at an appropriate stage see “Protective Groups in Organic Synthesis”, 3rd Edition (1999) by Greene and Wuts. For example compounds of formula I in which R³ represents a cyclohexyl group substituted by an amino may be prepared by deprotecting a compound of formula II in which R², R⁴, and n are as previously defined and R³ represents cyclohexyl substituted by a protected amino group, for example tert-butoxycarbonylamino. Deprotection may be carried out by methods known to those skilled in the art for example by acid hydrolysis for example using hydrochloric acid. Similarly compounds of formula I in which R² is hydroxy may be prepared by deprotecting a compound of formula II in which R² represents a protected hydroxy group, for example allyloxy. Deprotection may be carried out by methods known to those skilled in the art for example using tetrakis(triphenylphosphine)palladium optionally in the presence of a base for example morpholine in the presence of a solvent for example dichloromethane.

### Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-1 mg/kg body weight. Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is also provided a pharmaceutical formulation including any of the compounds of the invention, or pharmaceutically acceptable derivatives thereof, in admixture with pharmaceutically acceptable adjuvants, diluents and/or carriers.

### Pharmacological Properties

The compounds of formula (I) are useful for the treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

The compounds are also potentially useful for the prevention or treatment of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

The compounds are also potentially useful for the prevention or treatment of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

The compounds are also potentially useful for the treatment of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic shock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

The compounds are also potentially useful as agents in treatment of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders. The compounds are also potentially useful as agents in treatment of (esophageal) achalasia.

In another aspect the present invention provides a compound of formula I as previously defined for use as a medicament.

In a further aspect the present invention provides the use of a compound of formula I in the preparation of a medicament for the treatment or prophylaxis of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

In a further aspect the present invention provides the use of a compound of formula I in the preparation of a medicament for the treatment or prophylaxis of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

In a further aspect the present invention provides the use of a compound of formula I in the preparation of a medicament for the treatment or prophylaxis of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

In a further aspect the present invention provides the use of a compound of formula I in the preparation of a medicament for the treatment or prophylaxis of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic shock, stroke, cerebral apoplexy, cerebral infarction, cerebral ischaemia, cerebral thrombosis, cerebral embolism, cerebral hemorrhagia, metabolic is disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

In a further aspect the present invention provides the use of a compound of formula I in the preparation of a medicament for the treatment or prophylaxis of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders.

In a still further aspect the present invention provides a method comprising administering a pharmacologically effective amount of a compound of formula I to a patient in need thereof for the prophylaxis or treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

In a still further aspect the present invention provides a method comprising administering a pharmacologically effective amount of a compound of formula I to a patient in need thereof for the prophylaxis or treatment of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

In a still further aspect the present invention provides a method comprising administering a pharmacologically effective amount of a compound of formula I to a patient in need thereof for the prophylaxis or treatment of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma. In a still further aspect the present invention provides a method comprising administering a pharmacologically effective amount of a compound of formula I to a patient in need thereof for the prophylaxis or treatment of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic shock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

In a still further aspect the present invention provides a method comprising administering a pharmacologically effective amount of a compound of formula I to a patient in need thereof for the prophylaxis or treatment of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders.

The compounds of the present invention are particularly suitable for the treatment of obesity or being overweight, (e.g., promotion of weight loss and maintenance of weight loss), prevention or reversal of weight gain (e.g., rebound, medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items).

The compounds of formula (I) are useful for the treatment of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, and neurological disorders such as dementia, neurological disorders (e.g. Multiple Sclerosis), Raynaud's syndrome, Parkinson's disease, Huntington's chorea and Alzheimer's disease. The compounds are also potentially useful for the treatment of immune, cardiovascular, reproductive and endocrine disorders, septic shock and diseases related to the respiratory and gastrointestinal systems (e.g. diarrhea). The compounds are also potentially useful as agents in treatment of extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms. The compounds may also eliminate the increase in weight that normally accompanies the cessation of smoking.

In another aspect the present invention provides a compound of formula I as previously defined for use as a medicament.

In a further aspect the present invention provides the use of a compound of formula I in the preparation of a medicament for the treatment or prophylaxis of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms.

In a still further aspect the present invention provides a method of treating obesity, psychiatric disorders such as psychotic disorders such as schizophrenia and bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms comprising administering a pharmacologically effective amount of a compound of formula I to a patient in need thereof.

The compounds of the present invention are particularly suitable for the treatment of obesity, e.g. by reduction of appetite and body weight, maintenance of weight reduction and prevention of rebound.

The compounds of the present invention may also be used to prevent or reverse medication-induced weight gain, e.g. weight gain caused by antipsychotic (neuroleptic) treatment(s). The compounds of the present invention may also be used to prevent or reverse weight gain associated with smoking cessation.

The compounds of the present invention are suitable for use in treating the above indications in juvenile or adolescent patient populations.

The compounds of the present invention may also be suitable for use in the regulation of bone mass and bone loss and therefore useful in the treatment of osteoporosis and other bone diseases.

### Combination Therapy

The compounds of the invention may be combined with another therapeutic agent that is useful in the treatment of obesity such as other anti-obesity drugs, that affect energy expenditure, glycolysis, gluconeogenesis, glucogenolysis, lipolysis, lipogenesis, fat absorption, fat storage, fat excretion, hunger and/or satiety and/or craving mechanisms, appetite/motivation, food intake, or G-I motility.

The compounds of the invention may further be combined with another therapeutic agent that is useful in the treatment of disorders associated with obesity such as hypertension, hyperlipidaemias, dyslipidaemias, diabetes, sleep apnea, asthma, heart disorders, atherosclerosis, macro and micro vascular diseases, liver steatosis, cancer, joint disorders, and gallbladder disorders. For example, a compound of the present invention may be used in combination with a another therapeutic agent that lowers blood pressure or that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

The compounds of the invention may be used alongside other therapies for the treatment of obesity and its associated complications the metabolic syndrome and type 2 diabetes, these include biguanide drugs, insulin (synthetic insulin analogues) and oral antihyperglycemics (these are divided into prandial glucose regulators and alpha-glucosidase inhibitors). In another aspect of the invention, the compound of formula I, or a pharmaceutically acceptable salt thereof may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin.

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

The present invention also includes a compound of the present invention in combination with a bile acid sequestering agent, for example colestipol or cholestyramine or cholestagel.

According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration one or more of the following agents selected from:

a CETP (cholesteryl ester transfer protein) inhibitor;
a cholesterol absorption antagonist;
a MTP (microsomal transfer protein) inhibitor;
a nicotinic acid derivative, including slow release and combination products;
a phytosterol compound;
probucol;
an anti-coagulant;
an omega-3 fatty acid;
another anti-obesity compound for example sibutramine, phentermine, orlistat, bupropion, ephedrine, thyroxine;
an antihypertensive compound for example an angiotensin converting enzyme (ACE) inhibitor, an angiotensin II receptor antagonist, an adrenergic blocker, an alpha adrenergic blocker, a beta adrenergic blocker, a mixed alpha/beta adrenergic blocker, an adrenergic stimulant, calcium channel blocker, an AT-1 blocker, a saluretic, a diuretic or a vasodilator;
a melanin concentrating hormone (MCH) modulator;
an NPY receptor modulator;
an orexin receptor modulator;
a phosphoinositide-dependent protein kinase (PDK) modulator; or
modulators of nuclear receptors for example LXR, FXR, RXR, GR, ERRα, β, PPARα, β, γ and RORalpha;
a monoamine transmission-modulating agent, for example a selective serotonin reuptake inhibitor (SSRI), a noradrenaline reuptake inhibitor (NARI), a noradrenaline-serotonin reuptake inhibitor (SNRI), a monoamine oxidase inhibitor (MAOI), a tricyclic antidepressive agent (TCA), a noradrenergic and specific serotonergic antidepressant (NaSSA);
an antipsychotic agent for example olanzapine and clozapine;
a serotonin receptor modulator;
a leptin/leptin receptor modulator;
a ghrelin/ghrelin receptor modulator;
a DPP-IV inhibitor;
or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of very low calorie diets (VLCD) or low-calorie diets (LCD).

Therefore in an additional feature of the invention, there is provided a method for the treatment of obesity and its associated complications in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section or a pharmaceutically acceptable salt, solvate, solvate of such a salt or a prodrug thereof. According to a further aspect of the invention there is provided a pharmaceutical composition which comprises a compound of formula I, or a pharmaceutically acceptable salt thereof, and a compound from one of the other classes of compounds described in this combination section or a pharmaceutically acceptable salt, solvate, solvate of such a salt or a prodrug thereof, in association with a pharmaceutically acceptable diluent or carrier. According to a further aspect of the present invention there is provided a kit comprising a compound of formula I, or a pharmaceutically acceptable salt thereof, and a compound from one of the other classes of compounds described in this combination section or a pharmaceutically acceptable salt, solvate, solvate of such a salt or a prodrug thereof.

According to a further aspect of the present invention there is provided a kit comprising:

a) a compound of formula I, or a pharmaceutically acceptable salt thereof, in a first unit dosage form;
b) a compound from one of the other classes of compounds described in this combination section or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:

a) a compound of formula I, or a pharmaceutically acceptable salt thereof, together with a pharmaceutically acceptable diluent or carrier, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of obesity and its associated complications in a warm-blooded animal, such as man.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man. According to a further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment. Furthermore, a compound of the invention may also be combined with therapeutic agents that are useful in the treatment of disorders or conditions associated with obesity (such as type II diabetes, metabolic syndrome, dyslipidemia, impaired glucose tolerance, hypertension, coronary heart disease, non-alcoholic steatohepatitis, osteoarthritis and some cancers) and psychiatric and neurological conditions.

It will be understood that there are medically accepted definitions of obesity and being overweight. A patient may be identified by, for example, measuring body mass index (BMI), which is calculated by dividing weight in kilograms by height in metres squared, and comparing the result with the definitions.

### Pharmacological Activity

Compounds of the present invention are active against the receptor product of the CB1 gene. The affinity of the compounds of the invention for central cannabinoid receptors is demonstrable in methods described in Devane et al, Molecular Pharmacology, 1988, 34,605 or those described in WO01/70700 or EP 656354. Alternatively the assay may be performed as follows.

10 μg of membranes prepared from cells stably transfected with the CB1 gene were suspended in 200 μl of 100 mM NaCl, 5 mM MgCl₂, 1 mM EDTA, 50 mM HEPES (pH 7.4), 1 mM DTT, 0.1% BSA and 100 μM GDP. To this was added an EC80 concentration of agonist (CP55940), the required concentration of test compound and 0.1 μCi [³⁵S]-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 nM NaCl). Filters were then covered with scintillant and counted for the amount of [³⁵S]-GTPγS retained by the filter.

Activity is measured in the absence of all ligands (minimum activity) or in the presence of an EC80 concentration of CP55940 (maximum activity). These activities are set as 0% and 100% activity respectively. At various concentrations of novel ligand, activity is calculated as a percentage of the maximum activity and plotted. The data are fitted using the equation y=A+((B−A)/1+((C/x)UD)) and the IC50 value determined as the concentration required to give half maximal inhibition of GTPγS binding under the conditions used.

The compounds of the present invention are active at the CB1 receptor (IC50<1 micromolar). Most preferred compounds have IC50<200 nanomolar. For example the IC50 of Example 10 is 1.95 nM.

The compounds of the invention are believed to be selective CB1 antagonists or inverse agonists. The potency, selectivity profile and side effect propensity may limit the clinical usefulness of hitherto known compounds with alleged CB1 antagonistic/inverse agonistic properties. In this regard, preclinical evaluation of compounds of the present invention in models of gastrointestinal and/or cardiovascular function indicates that they offer significant advantages compared to representative reference CB1 antagonist/inverse agonist agents.

The compounds of the present invention may provide additional benefits in terms of potency, selectivity profile, bioavailability, half-life in plasma, blood brain permeability, plasma protein binding (for example higher free fraction of drug) or solubility compared to representative reference CB1 antagonists/inverse agonist agents.

The utility of the compounds of the present invention in the treatment of obesity and related conditions is demonstrated by a decrease in body weight in cafeteria diet-induced obese mice. Female C57B1/6J mice were given ad libitum access to calorie-dense ‘cafeteria’ diet (soft chocolate/cocoa-type pastry, chocolate, fatty cheese and nougat) and standard lab chow for 8-10 weeks. Compounds to be tested were then administered systemically (iv, ip, sc or po) once daily for a minimum of 5 days, and the body weights of the mice monitored on a daily basis. Simultaneous assessment of adiposity was carried by means of DEXA imaging at baseline and termination of the study. Blood sampling was also carried out to assay changes in obesity-related plasma markers.

### EXAMPLES

### Abbreviations

abs. absolute
AcOH acetic acid
aq aqueous
DCM dichloromethane
DMF dimethylformamide
DEA diethylamine
DEAD diethyl azodicarboxylate

### DIEA N,N-diisopropylethylamine

DMAP 4-dimethylaminopyridine
EtOAc ethyl acetate
Et₃N triethylamine

### Ex or EX Example

LiHMDS lithium hexamethyldisilazide
NH₄Ac ammonium acetate
Me methyl
MeOH methanol
MeCN acetonitrile
rt or RT room temperature
TEA triethylamine
THF tetrahydrofuran
t triplet
singlet
d doublet
q quartet
qvint quintet
m multiplet
br broad
bs broad singlet
dm doublet of multiplet
bt broad triplet
dd doublet of doublet

### General Experimental Procedures

Mass spectra were recorded on either a Micromass ZQ single quadrupole or a Micromass LCZ single quadrupole mass spectrometer both equipped with a pneumatically assisted electrospray interface (LC-MS). ¹H NMR measurements were performed on either a Varian Mercury 300 or a Varian Inova 500, operating at ¹H frequencies of 300 and 500 MHz respectively. Chemical shifts are given in ppm with CDCl₃ as internal standard. CDCl₃ is used as the solvent for NMR unless otherwise stated. Purification was performed on a semipreparative HPLC (High Performance Liquid Chromatography) with a mass triggered fraction collector, Shimadzu QP 8000 single quadrupole mass spectrometer equipped with 19×100 mm C8 column. The mobile phase used was, if nothing else is stated, acetonitrile and buffer (0.1 M ammonium acetate:acetonitrile 95:5).

For isolation of isomers, a Kromasil CN E9344 (250×20 mm i.d.) column was used.

Heptane:ethyl acetate:DEA 95:5:0.1 was used as mobile phase (1 ml/min). Fraction collection was guided using a UV-detector (330 nm).
Typical HPLC-parameters for purity analysis:

### HPLC-system: Agilent 1100

### Column: Zorbax Eclipse XDB-C8 150×4.6 mm

Time of analysis: 15 min
Flow: 1.5 ml/min
Mobilphase: A: water, 5% MeOH
B: MeOH

### Temperature: 40° C.

### Detector: Uv 240 nm

### EXAMPLE OF THE INVENTION

### Example 1

### 4-{-4-cyano-1-(2,4-dichlorophenyl)-3-[(piperidin-1-ylamino)carbonyl]-1H-pyrazol-5-yl}phenyl 3,3,3-trifluoropropane-1-sulfonate

### Step A: Ethyl chloro[(2,4-dichlorophenyl)hydrazono]acetate

Sodium nitrite (877 mg, 12.71 mmol) in water (5 ml) was added to a suspension of 2,4-dichloroaniline (2.0 g, 12.34 mmol) in 24% HCl (5 ml, aq) at 0° C. The reaction was continued at room temperature for 1 hour. A suspension of ethyl 2-chloro-3-oxobutanoate (2.03 g, 12.32 mmol) in 30% acetic acid (12 ml, aq) was added at 0° C. and the reaction continued at room temperature for 16 hours. The mixture was filtered and the filtrate washed with water, dissolved in DCM, washed with 5% NaHCO₃ (50 ml, aq) and water and dried over MgSO₄. The product was further purified by flash chromatography (SiO₂, toluene) to give a yellow powder (1.87 g, 51%).

¹H NMR (399.964 MHz) δ 8.65 (s, 1H), 7.48 (d, 1H), 7.27 (s, 1H), 7.18 (d, 1H), 4.36 (q, 2H), 1.37 (t, 3H).

### Step B: 3-[4-(benzyloxy)phenyl]-3-oxopropanenitrile

Acetonitrile (9.65 ml, 185.74 mmol) was added to a solution of N-butyllithium (2.5 M in hexane, 75 ml) in dry THF (30 ml) at −78° C. The reaction was continued at −78° C. for 20 minutes. A suspension of 4-benzyloxybenzoic acid methyl ester (15.00 g, 61.91 mmol) in dry THF/diethylether (4:1, 100 ml) was added over 20 minutes at −78° C. The reaction was continued at −78° C. for 30 minutes and then quenched with HCl (4M, 120 ml). The product was collected by filtration, washed with water and further purified by recrystallisation from ethanol (8.55 g, 55%).

¹H NMR (399.964 MHz) δ 7.86 (d, 2H), 7.44-7.34 (m, 5H), 7.03 (d, 2H), 5.13 (s, 2H), 3.98 (s, 2H).

### Step C: Ethyl 5-[4-(benzyloxy)phenyl]-4-cyano-1-(2,4-dichlorophenyl)-1H-pyrazole-3-carboxylate

Ethyl chloro[(2,4-dichlorophenyl)hydrazono]acetate (1.84 g, 6.24 mmol) and 3-[4-(benzyloxy)phenyl]-3-oxopropanenitrile (1.57 g, 6.24 mmol) were dissolved in ethanol (150 ml). Sodium ethoxide was added (3.5 ml, 21 wt % in ethanol) and the mixture boiled under reflux for 28 hours. The mixture was cooled to room temperature and the solvent evaporated. The mixture was redissolved in ethyl acetate, washed with water and dried over MgSO₄. The product was further purified by flash chromatography (SiO₂, toluene/ethyl acetate, product came at 3% ethyl acetate) and preparatory HPLC (kromasil C8 column, ammonium acetate (aq, 0.1 M):acetonitrile, product came at 100% acetonitrile) to give an almost white powder (403 mg, 13%).

¹H NMR (399.964 MHz) δ 7.45-7.20 (m, 10H), 6.93 (d, 2H), 5.03 (s, 2H), 4.49 (q, 2H), 1.43 (t, 3H). MS m/z 492, 494, 496 (M+H)⁺.

### Step D: 5-[4-(benzyloxo)phenyl]-4-cyano-1-(2,4-dichlorophenyl)-1H-pyrazole-3-carboxylic acid

Ethyl 5-[4-(benzyloxy)phenyl]-4-cyano-1-(2,4-dichlorophenyl)-1H-pyrazole-3-carboxylate (243 mg, 0.49 mmol) and sodium hydroxide (1.02 g, 25.41 mmol) were boiled under reflux in water/ethanol (1:5, 30 ml) for 2 hours. The solvent was evaporated and the mixture suspended in water and neutralised with HCl (conc.). The product was collected by filtration, washed with water and dried at reduced pressure (181 mg, crude).

¹H NMR (399.964 MHz) δ 8.20-6.80 (m, 12H), 5.00 (s, 2H). MS m/z 464, 466, 468 (M+H)⁺.

### Step E: 5-[4-(benzyloxy)phenyl]-4-cyano-1-(2,4-dichlorophenyl)-N-piperidin-1-yl-1H-pyrazole-3-carboxamide

A solution of oxalyl chloride (1 ml) in DCM (2 ml) was added to 5-[4-(benzyloxo)phenyl]-4-cyano-1-(2,4-dichlorophenyl)-1H-pyrazole-3-carboxylic acid (181 mg crude) in DCM (3 ml). One drop of DMF was added and the reaction continued at room temperature for 1 hour. The solvent and excess oxalyl chloride was evaporated off and the mixture suspended in 3 ml DCM and added to piperidin-1-amine hydrochloride (64 mg, 0.47 mmol) in DCM/K₂CO₃ (10%, aq) (2:3, 5 ml). The reaction was continued at room temperature for 3 hours. The phases were separated and the organic phase washed with water and dried over MgSO₄ (226 mg, crude).

¹H NMR (399.964 MHz) δ 7.60-7.10 (m, 1H), 6.92 (d, 2H), 5.03 (s, 2H), 2.96-2.80 (br, 4H), 1.84-1.68 (br, 4H), 1.58-1.30 (br, 2H). MS m/z 546, 548, 560 (M+H)⁺.

### Step F: 4-cyano-1-(2,4-dichlorophenyl)-5-(4-hydroxyphenyl)-N-piperidin-1-yl-1H-pyrazole-3-carboxamide

Dimethyl sulfide (440 μl, 6.0 mmol) and boron trifluoride diethyl etherate (740 μl, 6.0 mmol) were added to 5-[4-(benzyloxy)phenyl]-4-cyano-1-(2,4-dichlorophenyl)-N-piperidin-1-yl-1H-pyrazole-3-carboxamide (226 mg, crude) in DCM (5 ml). The reaction was continued at room temperature for 47 hours. Water was added and the phases separated. The organic phase was washed with water and then evaporated. The mixture was stirred in methanol at room temperature for 3 hours. Water was added, the methanol evaporated and the water extracted with diethyl ether. The organic phase washed with water and dried over MgSO₄ (146 mg, crude).

¹H NMR (399.964 MHz) δ 7.45-6.30 (m, 4H), 7.09 (d, 2H), 6.82 (d, 2H), 2.93-2.83 (br, 4H), 1.73-1.59 (br, 4H), 1.43-1.32 (br, 2H). MS m/z 456, 458, 460 (M+H)⁺.

### Step G: 4-{4-cyano-1-(2,4-dichlorophenyl)-3-[(piperidin-1-ylamino)carbonyl]-1H-pyrazol-5-yl}phenyl 3,3,3-trifluoropropane-1-sulfonate

3,3,3-trifluoropropane-1-sulfonyl chloride (90 mg, 0.46 mmol) was added to a mixture of 4-cyano-1-(2,4-dichlorophenyl)-5-(4-hydroxyphenyl)-N-piperidin-1-yl-1H-pyrazole-3-carboxamide (146 mg, crude) and TEA (125 μl, 0.90 mmol) in DCM (5 ml) at −78° C., under N₂(g). The reaction was continued at −78° C. for 1 hour. Water was added, the phases were separated and the organic phase was washed with water. The product was further is purified by preparatory HPLC (kromasil C8 column, ammonium acetate (aq, 0.1 M):acetonitrile, product came at 97% acetonitrile) to give an almost white powder (93 mg, 30% yield for 4 steps). ¹H NMR (399.964 MHz) δ 7.60-7.20 (m, 8H), 3.54-3.44 (m, 2H), 2.90-2.80 (br, 4H), 2.80-2.65 (m, 2H), 1.76-1.66 (br, 4H), 1.46-1.36 (br, 2H).

HRMS Calcd for [C₂₅H₂₂Cl₂F₃N₅O₄S+H]⁺: 616.080. Found: 616.084.

### Example 2

### 4-{4-cyano-1-(2,4-dichlorophenyl)-3-[(piperidin-1-ylamino)carbonyl]-1H-pyrazol-5-yl}phenyl 3-methylbutane-1-sulfonate

3-methylbutane-1-sulfonyl chloride (80 mg, 0.47 mmol) was added to a mixture of 4-cyano-1-(2,4-dichlorophenyl)-5-(4-hydroxyphenyl)-N-piperidin-1-yl-1H-pyrazole-3-carboxamide, prepared as in Ex. 1, Step F (113 mg, crude) and TEA (70 μl, 0.50 mmol) in DCM (5 ml) at −78° C. under N₂(g). The reaction was continued at −78° C. for 1.5 hour. Water was added and the phases were separated. The organic phase was washed with water and dried over MgSO₄. The product was further purified by preparatory HPLC (kromasil C8 column, ammonium acetate (aq, 0.1 M):acetonitrile, product came at 100% acetonitrile) to give an almost white powder (100 mg, 52% yield for 4 steps).

¹H NMR (399.964 MHz) δ 7.60-7.20 (m, 8H), 3.25-3.15 (m, 2H), 2.90-2.80 (br, 4H), 1.84-1.75 (m, 2H), 1.75-1.64 (m, 5H), 1.44-1.34 (br, 2H), 0.90 (d, 6H).

HRMS Calcd for [C₂₇H₂₉Cl₂N₅O₄S+H]⁺: 590.140. Found: 590.137.

### Example 3

### 4-[1-(2,4-dichlorophenyl-3-({[(1R,2S)-2-hydroxycyclohexyl]amino}carbonyl)-4-methyl-1H-pyrazol-5-yl]phenyl 3,3,3-trifluoropropane-1-sulfonate and 4-[1-(2,4-dichlorophenyl)-3-({[(1S,2R)-2-hydroxycyclohexyl]amino}carbonyl)-4-methyl-1H-pyrazol-5-yl]phenyl 3,3,3-trifluoropropane-1-sulfonate

### Step A 1-(4-Benzyloxy-phenyl)-propane-1-one

4-Hydroxypropiophenone (15.0 g, 0.10 mol) was dissolved in acetone (200 ml) together with potassium carbonate (13.8 g, 0.10 mol). Benzyl bromide (17.1 g, 0.10 mol) was added to and the reaction mixture boiled under reflux overnight. After cooling to room temperature the mixture was filtered and concentrated on the rotary evaporator to afford 24.0 g (100%) of the title compound as a white solid

### Step B 1-(4-Benzyloxyphenyl)-2-bromo-propane-1-one

1-(4-Benzyloxyphenyl)propane-1-one (4.80 g, 20.0 mmol) was suspended in acetic acid is (25 ml) and cooled to 0° C. Bromine (3.20 g, 20.0 mmol) was added dropwise and the reaction mixture was stirred for two hours at room temperature at which point the reaction mixture was a clear, yellow solution. After cooling, water (100 ml) was added and the product extracted with ether (2×100 ml). The combined organic extracts were washed with water, sodium hydrogen carbonate (caution! Gas evolves) and brine. The organic phase was dried (Na₂SO₄), filtered and evaporated leaving the title compound as a pale yellow solid (6.17 g, 97%).

### Step C 2-[2-(4-Benzyloxyphenyl)-2-oxoethyl]-3-oxo-butyric acid ethyl ester

A solution of sodium ethoxide was generated from sodium metal (0.53 g, 23.0 mmol) in 30 ml abs. ethanol. To this solution was added ethyl acetoacetate (3.00 g, 23.0 mmol) at 0° C. After 30 min. this solution was added to a solution of 1-(4-Benzyloxy-phenyl)-2-bromo-propane-1-one (6.17 g, 19.0 mmol) in ethanol:toluene (30:15 ml) and the reaction mixture stirred overnight. Acidic work-up with 1 M HCl, extraction with ethyl acetate (3×), washing with brine, drying (Na₂SO₄), filtering and evaporation left a crude product purified by flash chromatography (hexane:EtOAc 95:5-70:30) affording 5.18 g of the title compound as a pale yellow oil.

### Step D 5-(4-Benzyloxyphenyl)-1-(2,4-dichlorophenol)-4-methyl-1H-pyrazole-3-carboxylic acid

A solution of sodium ethoxide was generated from sodium metal (0.19 g, 8.26 mmol) in 20 ml abs. ethanol. To this solution was added 2-[2-(4-benzyloxy-phenyl)-2-oxoethyl]-3-oxo-butyric acid ethyl ester (2.13 g, 6.00 mmol) and the reaction mixture stirred at room temperature for 30 min. A previously prepared solution of 2,4-dichlorodiazonium chloride (prepared from 2,4-dichloroaniline (1.19 g, 7.30 mmol) in 3 ml 24% HCl and sodium nitrite (0.52 g, 7.50 mmol) in 3 ml water at 0° C.) was added in 5 portions keeping the temperature below 5° C. After stirring at room temperature for 2.5 hours water was added, the product extracted with EtOAc (3×). The combined organic extracts was dried (Na₂SO₄), filtered and evaporated. The residue was dissolved in ethanol (40 ml) and sodium hydroxide (0.80 g, 20.0 mmol) in 10 ml of water was added. After 2 hours boiling under reflux the reaction mixture was cooled, acidified with HCl and the product extracted with EtOAc (3×). After washing, drying (Na₂SO₄), filtration and concentration, the residue was purified by flash chromotography (hexane:EtOAc 70:30-50:50) affording 1.84 g (68%) of the title compound as a pale yellow solid.

### Step E: 5-[4-(benzyloxy)phenyl]-1-(2,4-dichlorophenyl)-N-[(1R,2S)-2-hydroxycyclohexyl]-4-methyl-1H-pyrazole-3-carboxamide and 5-[4-(benzyloxy)phenyl]-1-(2,4-dichlorophenyl)-N-[(1S,2R)-2-hydroxycyclohexyl]-4-methyl-1H-pyrazole-3-carboxamide

Oxalyl chloride (1 ml) was added to 5-[4-(benzyloxy)phenyl]-1-(2,4-dichlorophenyl)-4-methyl-1H-pyrazole-3-carboxylic acid (500 mg, 1.10 mmol) in DCM (5 ml). One drop of DMF was added and the reaction continued at room temperature for 1 hour. The solvent and excess oxalyl chloride was evaporated and the mixture suspended in 3 ml DCM and added to cis-2-aminocyclohexanol hydrochloride (204 mg, 1.35 mmol) in DCM/K₂CO₃ (10%, aq) (2:4, 6 ml). The reaction was continued at room temperature for 2 hours. The phases were separated and the organic phase washed with water and dried over MgSO₄ (610 mg, crude).

¹H NMR (499.961 MHz) δ 7.50-7.25 (m, 9H), 7.08 (d, 2H), 6.94 (d, 2H), 5.05 (s, 2H), 4.20-4.10 (br, 1H), 4.05-4.00 (br, 1H), 3.10-2.85 (br, 1H), 2.39 (s, 3H), 1.84-1.56 (m, 6H), 1.50-1.36 (br, 2H). MS m/z 550, 552, 554 (M+H)⁺.

### Step F: 1-(2,4-dichlorophenyl)-N-[(1R,2S)-2-hydroxycyclohexyl]-5-(4-hydroxyphenyl)-4-methyl-1H-pyrazole-3-carboxamide and 1-(2,4-dichlorophenyl)-N-[(1S,2R)-2-hydroxycyclohexyl]-5-(4-hydroxyphenyl)-4-methyl-1H-pyrazole-3-carboxamide

Dimethyl sulfide (813 μl, 11.08 mmol) and boron trifluoride diethyl etherate (1.40 ml, 11.05 mmol) were added to a mixture of 5-[4-(benzyloxy)phenyl]-1-(2,4-dichlorophenyl)-N-[(1R,2S)-2-hydroxycyclohexyl]-4-methyl-1H-pyrazole-3-carboxamide and 5-[4-(benzyloxy)phenyl]-1-(2,4-dichlorophenyl)-N-[(1S,2R)-2-hydroxycyclohexyl]-4-methyl-1H-pyrazole-3-carboxamide (610 mg, crude) in DCM (5 ml). The reaction was continued at room temperature for 40 hours. Water was added and the phases separated. The organic phase was washed with water and dried over MgSO₄ (531 mg, crude).

¹H NMR (399.964 MHz) δ 9.00-8.00 (br, 1H), 7.42-7.32 (m, 2H), 7.30-7.18 (m, 2H), 6.90 (d, 2H), 6.78 (d, 2H), 4.17-4.07 (br, 1H), 4.03-3.96 (br, 1H), 4.00-3.00 (br, 1H), 2.31 (s, 3H), 1.80-1.50 (m, 6H), 1.45-1.30 (br, 2H). MS m/z 460, 462, 464 (M+H)⁺.

### Step G: 4-[1-(2,4-dichlorophenyl)-3-({[(1R,2S)-2-hydroxycyclohexyl]amino}carbonyl-4-methyl-1H-pyrazol-5-yl]phenyl 3,3,3-trifluoropropane-1-sulfonate and 4-[1-(2,4-dichlorophenyl)-3-({[(1S,2R)-2-hydroxycyclohexyl]amino}carbonyl)-4-methyl-1H-pyrazol-5-yl]phenyl 3,3,3-trifluoropropane-1-sulfonate

3,3,3-trifluoropropane-1-sulfonyl chloride (59 mg, 0.30 mmol) was added to a mixture of TEA (50 μl, 0.35 mmol), racemic mixture of 1-(2,4-dichlorophenyl)-N-[(1R,2S)-2-hydroxycyclohexyl]-5-(4-hydroxyphenyl)-4-methyl-1H-pyrazole-3-carboxamide and 1-(2,4-dichlorophenyl)-N-[(1S,2R)-2-hydroxycyclohexyl]-5-(4-hydroxyphenyl)-4-methyl-1H-pyrazole-3-carboxamide (139 mg, crude) in DCM (5 ml) at −78° C., under N₂(g). The reaction was continued at −78° C. for 1 hour. Water was added and the phases were separated. The organic phase was washed with water and dried over MgSO₄. The product was further purified by preparatory HPLC (kromasil C8 column, ammonium acetate (aq, 0.1 M):acetonitrile, product came at 98% acetonitrile) to give an almost white powder (103 mg, 57% yield for 3 steps).

¹H NMR (399.964 MHz) δ 7.39 (s, 1H), 7.30-7.15 (m, 7H), 4.17-4.07 (m, 1H), 4.05-3.99 (m, 1H), 3.50-3.43 (m, 2H), 2.84-2.70 (m, 2H), 2.35 (s, 3H), 2.05-1.90 (br, 1H), 1.80-1.50 (m, 6H), 1.50-1.35 (m, 2H).

HRMS Calcd for [C₂₆H₂₆Cl₂F₃N₃O₅S+H]⁺: 620.100. Found: 620.101.

### Example 4

### 4-(1-(2,4-dichlorophenyl)-3-{[(5-fluoropyridin-2-yl)amino]carbonyl}-4-methyl-1H-pyrazol-5-yl)phenyl 3,3,3-trifluoropropane-1-sulfonate

### Step A: 5-[4-(benzyloxy)phenyl]-1-(2,4-dichlorophenyl)-N-(5-fluoropyridin-2-yl)-4-methyl-1H-pyrazole-3-carboxamide

Benzotriazol-1-yl-oxytripyrrolidinophosphonium hexafluorophosphate (252 mg, 0.48 mmol) in DCM (1 ml) was added to a suspension of 5-[4-benzyloxy)phenyl]-1-(2,4-dichlorophenyl)-4-methyl-1H-pyrazole-3-carboxylic acid (200 mg, 0.44 mmol), 5-fluoropyridin-2-amine (57 mg, 0.51 mmol) and TEA (61 μl, 0.44 mmol) in DCM (4 ml) at 0° C. The reaction was continued at 0° C. for 15 minutes and then at room temperature for 72 hours. Water was added and the phases separated. The organic phase was washed with water and dried over MgSO₄. The product was further purified by flash chromatography (SiO₂, toluene/ethyl acetate, product came at 2% ethyl acetate) (165 mg, 68%).

¹H NMR (399.964 MHz) δ 9.48 (s, 1H), 8.44-8.34 (m, 1H), 8.15-8.10 (m, 1H), 7.50-7.10 (m, 9H), 7.06 (d, 2H), 6.91 (d, 2H), 5.01 (s, 2H), 2.41 (s, 3H).

MS m/z 547, 549, 551 (M+H)⁺.

### Step B: 1-(2,4-dichlorophenyl)-N-(5-fluoropyridin-2-yl)-5-(4-hydroxyphenyl)-4-methyl-1H-pyrazole-3-carboxamide

Dimethyl sulfide (221 μl, 3.01 mmol) and boron trifluoride diethyl etherate (382 μl, 3.01 mmol) were added to 5-[4-(benzyloxy)phenyl]-1-(2,4-dichlorophenyl)-N-(5-fluoropyridin-2-yl)-4-methyl-1H-pyrazole-3-carboxamide (165 mg, 0.301 mmol) in DCM (5 ml). The reaction was continued at room temperature for 67 hours. Water was added and the phases separated. The organic phase was washed with water and then evaporated. The mixture was stirred in methanol at room temperature for 24 hours. Water was added, the methanol evaporated and the water extracted with diethyl ether. The organic phase washed with water and dried over MgSO₄ (142 mg, crude). ¹H NMR (399.964 MHz) δ 9.50 (s, 1H), 8.50-6.20 (m, 10H), 2.43 (s, 3H). MS m/z 457, 459, 462 (M+H)⁺.

### Step C: 4-(1-(2,4-dichlorophenyl-3-{[(5-fluoropyridin-2-yl)amino]carbonyl}-4-methyl-1H-pyrazol-5-yl)phenyl 3,3,3-trifluoropropane-1-sulfonate

3,3,3-trifluoropropane-1-sulfonyl chloride (82.5 mg, 0.42 mmol) was added to a mixture of 1-(2,4-dichlorophenyl)-N-(5-fluoropyridin-2-yl)-5-(4-hydroxyphenyl)-4-methyl-1H-pyrazole-3-carboxamide (142 mg, crude) and TEA (50 μl, 0.35 mmol) in DCM (2 ml) at −78° C., under N₂(g). The reaction was continued at −78° C. for 2 hours. Water was added and the phases were separated. The organic phase was washed with water and dried over MgSO₄. The product was further purified by preparatory HPLC (kromasil C8 column, ammonium acetate (aq, 0.1 M):acetonitrile, product came at 100% acetonitrile) to give an almost white powder (111 mg, 60% yield for 2 steps). ¹H NMR (399.964 MHz) δ 9.43 (s, 1H), 8.40-8.33 (m, 1H), 8.15-8.10 (m, 1H), 7.48-7.40 (m, 2H), 7.33-7.26 (m, 2H), 7.26-7.17 (m, 4H), 3.51-3.43 (m, 2H), 2.85-2.70 (m, 2H), 2.41 (s, 3H). HRMS Calcd for [C₂₅H₁₈Cl₂F₄N₄O₄S+H]⁺: 617.044. Found: 617.047.

### Example 5

### 4-(1-(2,4-dichlorophenyl)-3-{[(3,4-difluoro-2-hydroxyphenyl)amino]carbonyl}-4-methyl-1H-pyrazol-5-yl)phenyl 3,33-trifluoropropane-1-sulfonate

### Step A: 5-[4-(benzyloxy)phenyl]-1-(2,4-dichlorophenyl)-N-(3,4-difluoro-2-hydroxyphenyl)-4-methyl-1H-pyrazole-3-carboxamide

Oxalyl chloride (1 ml) was added to 5-[4-(benzyloxy)phenyl]-1-(2,4-dichlorophenyl)-4-methyl-1H-pyrazole-3-carboxylic acid (300 mg, 0.66 mmol) in DCM (5 ml). One drop of DMF was added and the reaction continued at room temperature for 4 hours. The solvent and excess oxalyl chloride was evaporated and the mixture suspended in 3 ml DCM and added to 6-amino-2,3-difluorophenol (162 mg, 1.12 mmol) in DCM/K₂CO₃(10%, aq) (2:3, 5 ml). The reaction was continued at room temperature for 19 hours. DMAP (50 mg, 0.41 mmol) was added and the reaction continued at room temperature for 5 hours. The phases were separated and the organic phase washed with water and dried over MgSO₄ (435 mg, crude). ¹H NMR (399.964 MHz) δ 10.00-9.60 (br, 1H), 9.04 (s, 1H), 7.45-6.58 (m, 14H), 5.02 (s, 2H), 2.37 (s, 3H). MS m/z 580, 582, 584 (M+H)⁺.

### Step B: 1-(2,4-dichlorophenyl)-N-(3,4-difluoro-2-hydroxyphenyl)-5-(4-hydroxyphenyl)-4-methyl-1H-pyrazole-3-carboxamide

Dimethyl sulfide (275 μl, 3.75 mmol) and boron trifluoride diethyl etherate (475 μl, 3.75 mmol) were added to 5-[4-(benzyloxy)phenyl]-1-(2,4-dichlorophenyl)-N-(3,4-difluoro-2-hydroxyphenyl)-4-methyl-1H-pyrazole-3-carboxamide (435 mg, crude) in DCM (5 ml). The reaction was continued at room temperature for 86 hours. Water was added and the phases separated. The organic phase was washed with water and dried over MgSO₄ (325 mg, crude). ¹H NMR (399.964 MHz) δ 9.85-9.65 (br, 1H), 9.03 (s, 1H), 7.45-6.55 (m, 10H), 2.34 (s, 3H). MS m/z 490, 492, 494 (M+H)⁺.

### Step C: N-[2-(allyloxy)-3,4-difluorophenyl]-1-(2,4-dichlorophenyl)-5-(4-hydroxyphenyl)-4-methyl-1H-pyrazole-3-carboxamide

Allyl bromide (44 μl, 0.52 mmol) was added to 1-(2,4-dichlorophenyl)-N-(3,4-difluoro-2-hydroxyphenyl)-5-(4-hydroxyphenyl)-4-methyl-1H-pyrazole-3-carboxamide (256 mg, crude) and cesium carbonate (170 mg, 0.52 mmol) in acetonitrile (6 ml). The reaction was continued at room temperature for 23 hours. Water and DCM were added, the phases separated and the organic phase washed with water and dried over MgSO₄ (196 mg, crude). ¹H NMR (399.964 MHz) δ 9.39 (s, 1H), 8.26-8.16 (m, 1H), 7.50-6.60 (m, 8H), 6.08-5.94 (m, 1H), 5.35 (d, 1H), 5.12 (d, 1H), 4.64 (d, 2H), 2.38 (s, 3H). MS m/z 530, 532, 534 (M+H)⁺.

### Step D: 4-[3-({[2-(allyloxy)-3,4-difluorophenyl]amino}carbonyl)-1-(2,4-dichlorophenyl)-4-methyl-1H-pyrazol-5-yl]phenyl 3,3,3-trifluoropropane-1-sulfonate

3,3,3-trifluoropropane-1-sulfonyl chloride (75 mg, 0.38 mmol) was added to a mixture of N-[2-(allyloxy)-3,4-difluorophenyl]-1-(2,4-dichlorophenyl)-5-(4-hydroxyphenyl)-4-methyl-1H-pyrazole-3-carboxamide (196 mg, crude) and TEA (50 μl, 0.35 mmol) in DCM (2 ml) at −78° C., under N₂(g). The reaction was continued at −78° C. for 3 hours. Water was added and the phases were separated. The organic phase was washed with water and dried over MgSO₄ (249 mg, crude).

¹H NMR (399.964 MHz) δ 9.37 (s, 1H), 8.28-8.18 (m, 1H), 7.50-6.80 (m, 8H), 6.08-5.92 (m, 1H), 5.35 (d, 1H), 5.11 (d, 1H), 4.64 (d, 2H), 3.51-3.43 (m, 2H), 2.90-2.70 (m, 2H), 2.38 (s, 3H). MS m/z 690, 692, 694 (M+H)⁺.

### Step E: 4-(1-(2,4-dichlorophenyl)-3-{[(3,4-difluoro-2-hydroxyphenyl)amino]carbonyl}-4-methyl-1H-pyrazol-5-yl)phenyl 3,3,3-trifluoropropane-1-sulfonate

Morpholine (350 μl) was added to 4-[3-({[2-(allyloxy)-3,4-difluorophenyl]amino}carbonyl)-1-(2,4-dichlorophenyl)-4-methyl-1H-pyrazol-5-yl]phenyl 3,3,3-trifluoropropane-1-sulfonate (249 mg, crude) and tetrakis(triphenylphosphine)palladium (90 mg, 0.08 mmol) in DCM (3 ml) under N₂(g). The reaction was continued at room temperature under N₂(g) for 3 hours. Water was added and the phases were separated. The organic phase was washed with water and dried over MgSO₄. The product was further purified by preparatory HPLC (kromasil C8 column, ammonium acetate (aq, 0.1 M):acetonitrile, product came at 100% acetonitrile) to give an almost white powder (77 mg, 23% yield for five steps). ¹H NMR (399.964 MHz) δ 9.55 (s, 1H), 8.96 (s, 1H), 7.46-7.42 (m, 1H), 7.35-7.18 (m, 6H), 6.90-6.83 (m, 1H), 6.70-6.60 (m, 1H), 3.53-3.45 (m, 2H), 2.85-2.71 (m, 2H), 2.38 (s, 3H). HRMS Calcd for [C₂₆H₁₈Cl₂F₅N₃O₅S+H]⁺: 650.034. Found: 650.038.

### Example 6

### 4-{1-(2,4-dichlorophenyl)-4-methyl-3-[(piperidin-1-ylamino)carbonyl]-1H-pyrazol-5-yl}phenyl 3-fluoropropane-1-sulfonate

A solution of 3-fluoropropane-1-sulfonyl chloride (160 mg, 1.00 mmol) in DCM (1.5 ml) was added to 1-(2,4-dichlorophenyl)-5-(4-hydroxyphenyl)-4-methyl-N-piperidin-1-yl-1H-pyrazole-3-carboxamide (200 mg, 0.45 mmol) and TEA (100 μl, 0.72 mmol) in DCM (1.5 ml) at −78° C. under N₂(g). The reaction was continued at −78° C. under N₂(g) for 3 hours. Water was added and the phases were separated. The organic phase was washed with water and dried over MgSO₄. The product was further purified by preparatory HPLC (kromasil C8 column, ammonium acetate (aq, 0.1 M):acetonitrile, product came at 97% acetonitrile) to give an almost white powder (191 mg, 74% yield). ¹H NMR (399.964 MHz) δ 7.70-7.55 (br, 1H), 7.38 (s, 1H), 7.28-7.10 (m, 6H), 4.57 (dt, 2H), 3.42-3.34 (m, 2H), 2.87-2.77 (m, is 4H), 2.32 (s, 3H), 2.40-2.22 (m, 2H), 1.76-1.66 (m, 4H), 1.45-1.33 (m, 2H). HRMS Calcd for [C₂₅H₂₇Cl₂FN₄O₄S+H]⁺: 569.119. Found: 569.119.

### Example 7

### 3,3,3-Trifluoropropane-1-sulfonic acid 4-[2-(2,4-dichlorophenyl)-5-(4-hydroxypiperidin-1-ylcarbamoyl)-4-methyl-2H-pyrazol-3-yl]phenyl ester

### Step A: 5-(4-Benzyloxyphenyl)-1-(2,4-dichlorophenyl)-4-methyl-1H-pyrazole-3-carboxylic acid (4-hydroxypiperidin-1-yl)amide

5-(4-Benzyloxyphenyl)-1-(2,4-dichlorophenyl)-4-methyl-1H-pyrazole-3-carboxylic acid (750 mg, 1.65 mmol, 1 equiv.) and thionyl chloride (20 equiv) were mixed and the resulting mixture was boiled under reflux for 3.5 h. Excess SOCl₂ was removed under reduced pressure and the residue was azeotroped with toluene to give the acid chloride. 4-Hydroxy-1-aminopiperidine (2 equiv.) was mixed with dichloromethane (10 ml) and THF (7 ml) and triethylamine (5 equiv). The mixture was cooled to −30° C. under a nitrogen atmosphere. A THF (5 ml) mixture of the acid chloride from above was added dropwise during 20 minutes. The resulting mixture was allowed to slowly warm to room temperature and stirred overnight. Aqueous NaOH (1M, 3 ml) was added and the mixture was left for 15 minutes. The reaction mixture was then diluted to 50 ml with dichloromethane and washed with water (2×20 ml) and brine (20 ml). The organic layer was dried (Na₂SO₄), filtered and concentrated under reduced pressure. The residue was purified by Horizon flash chromatography, 8% methanol in dichloromethane. The product fraction was concentrated under reduced pressure to give the title compound (506 mg 55% yield) as a solid). ¹H-NMR (CDCl₃): 1.72-1.83 (m, 2H), 1.93-2.02 (m, 2H), 2.32 (s, 3H), 2.75-2.84 (m, 2H), 3.04-3.13 (m, 2H), 3.74-3.82 (m, 1H), 5.00 (s, 2H), 6.87 (d, 2H), 7.00 (d, 2H), 7.20-7.41 (m, 8H), 7.66 (s, 1H). MS: 551 (M+1).

### Step B: 1-(2,4-Dichlorophenyl)-5-(4-hydroxyphenyl-4-methyl-1H-pyrazole-3-carboxylic acid (4-hydroxypiperidin-1-yl)amide

5-(4-Benzyloxyphenyl)-1-(2,4-dichlorophenyl)-4-methyl-1H-pyrazole-3-carboxylic acid (4-hydroxy-piperidin-1-yl)-amide (475 mg, 0.86 mmol, 1 equiv.) and Me₂S (5 equiv) were mixed in dichloromethane under a nitrogen atmosphere. BF₃xOEt₂ (5 equiv.) was added dropwise and the resulting mixture was stirred for 6 days at ambient temperature while continuously adding small volumes of dichloromethane and 1,4-dioxane. Methanol and water were then added and the mixture was stirred for 30 mins and was then concentrated under reduced pressure. The residue was extracted with ethyl acetate (3×50 ml). The organic layer was washed with brine (20 ml) and was then dried (Na₂SO₄), filtered and concentrated under reduced pressure. The crude material was purified by Horizon flash chromatography (8% methanol in dichloromethane) to give the title compound (304 mg, 76%) as a white solid. MS: 461 (M+1).

### Step C: 3,3,3-Trifluoropropane-1-sulfonic acid 4-[2-(2,4-dichlorophenyl)-5-(4-hydroxy-piperidin-1-ylcarbamoyl)-4-methyl-2H-pyrazol-3-yl]-phenyl ester

1-(2,4-Dichlorophenyl)-5-(4-hydroxyphenyl)-4-methyl-1H-pyrazole-3-carboxylic acid (4-hydroxy-piperidin-1-yl)-amide (99 mg, 0.21 mmol, 1 equiv.) was dissolved in dichloromethane (15 ml), THF (15 ml) and NEt₃ (3 equiv.) under nitrogen atmosphere. The solution was cooled to −78 and a solution of 3,3,3-trifluoro-propane-1-sulfonyl chloride in dichloromethane (1 ml) was added slowly while monitoring the progress with LC-MS. The reaction mixture was quenched by addition of methanol and water. The reaction mixture was concentrated under reduced pressure. The residue was purified by reverse phase HPLC (Kromasil C8, 5-100% acetonitrile in water with 0.1M ammonium acetate). The product fraction was freeze-dried to give the title compound (36 mg, 27%) as a white powder.

¹H-NMR (CDCl₃): 1.74-1.85 (m, 2H), 1.95-2.04 (m, 2H), 2.35 (s, 3H), 2.70-2.86 (m, 4H), 3.06-3.14 (m, 2H), 3.43-3.50 (m, 2H), 3.76-3.85 (m, 1H), 7.12-7.40 (m, 7H), 7.66 (s, 1H). HRMS: Calculated for [C₂₅H₂₅Cl₂F₃N₄O₅S+H⁺] 621.0953, found 621.0939

HPLC-UV: 98%

### Example 8

### 3,3,3-Trifluoro-propane-1-sulfonic acid 4-[2-(2,4-dichlorophenyl)-5-(3-hydroxy-piperidin-1-ylcarbamoyl)-4-methyl-2H-pyrazol-3-yl]phenyl ester

### Step A: 5-(4-Benzyloxyphenyl)-1-(2,4-dichlorophenyl)-4-methyl-1H-pyrazole-3-carboxylic acid (3-hydroxy-piperidin-1-yl)amide

This compound was prepared as described in Ex. 7, Step A using 3-hydroxy-1-aminopiperidine instead of 4-hydroxy-1-aminopiperidine. The title compound was obtained as a semisolid, 518 mg (48%).

MS: 551 (M+1).

### Step B: 1-(2,4-Dichlorophenyl)-5-(4-hydroxyphenyl)-4-methyl-1H-pyrazole-3-carboxylic acid (3-hydroxy-piperidin-1-yl)amide

This compound was prepared from the product of Step A above by the method described in Ex. 7, Step B and was obtained as a yellow oil, 385 mg (89%). MS: 461 (M+1).

### Step C: 3,3,3-Trifluoropropane-1-sulfonic acid 4-[2-(2,4-dichlorophenol)-5-(3-hydroxy-piperidin-1-ylcarbamoyl)-4-methyl-2H-pyrazol-3-yl]phenyl ester

This compound was prepared, in a similar manner to that described in Ex. 7, Step C, as a white solid, 37 mg (24%). after freeze-drying. ¹H-NMR (MeOH-d₄): 1.30-1.43 (m, 1H), 1.60-1.93 (3H), 2.29-2.37 (3H), 2.59-2.96 (m, 5H), 3.08-3.16 (m, 1H), 3.66-3.76 (m, 2H), 3.81-3.91 (m, 1H), 7.30-7.38 (m, 4H), 7.42-7.49 (1H), 7.52-7.59 (m, 2H). HRMS: Calculated for [C₂₅H₂₅Cl₂F₃N₄O₅S+H⁺] 621.0953, found 621.0947. HPLC-UV: 99%

### Example 9

### 3-Methyl-butane-1-sulfonic acid 4-[2-(2,4-dichlorophenyl)-4-methyl-5-(5-methyl-pyridin-2-ylcarbamoyl)-2H-pyrazol-3-1]phenyl ester or 4-(1-(2,4-dichlorophenyl)-4-methyl-3-{[(5-methylpyridin-2-yl)amino]carbonyl}-1H-pyrazol-5-yl)phenyl 3-methylbutane-1-sulfonate

### Step A: 4-benzyloxy propiophenone

To a solution of 4-hydroxy propiophenone (50 g, 0.3329 mol) in dry acetone (500 ml) was added benzyl bromide (56.94 g, 0.333 mol) followed by anh. K₂CO₃ (91.8 g, 0.665 mol). Reaction mixture was refluxed for 18 h, cooled to RT, filtered and filtrate was concentrated to yield 4-benzyloxy propiophenone (75 g, 93%) as a white solid.

### Step B Lithium 1-(4-Benzyloxy-phenyl)-3-ethoxycarbonyl-2-methyl-3-oxopropen-1-ol

To a solution of 4-benzyloxypropiophenone (50 g, 0.2083 mol) in dry THF (500 ml) at 0° C. was added LiHMDS (1M solution in THF, 208.3 ml) drop wise over a period of 1 h under N₂ atm. The reaction mixture was stirred at 0° C. for 1 hr. Added diethyl oxalate (33.49 g, 0.2296 mol) drop wise. Reaction mixture was allowed to warm to RT and stirred at RT for 16 hrs under N₂ atm. Reaction mixture was concentrated in the rotary evaporator at RT. To the residue was added dry diethyl ether (1 L) and the solid was filtered, washed with dry ether, and dried under vacuum to yield lithium salt of the diketoester (50 g) as yellow solid.

### Step C: 4-(4-Benzyloxyphenyl)-4-[(2,4-dichloro-phenyl)hydrazono]-3-methyl-2-oxo-butyric acid ethyl ester

A mixture of lithium salt from step 2 (50 g, 0.1461 mol) and 2,4-dichlorophenylhydrazine hydrochloride (34.33 g, 0.1608 mol) in ethanol (500 ml) was stirred at RT under N₂ atm for 18 hrs. The precipitate was filtered, washed with dry ether and dried under vacuum to yield hydrazone intermediate (35 g).

### Step D: ethyl 5-[4-(benzyloxy)phenyl]-1-(2,4-dichlorophenyl)-4-methyl-1H-pyrazole-3-carboxylate

Hydrazone intermediate (35 g) was dissolved in acetic acid (250 ml) and heated under reflux for 18 hrs. Reaction mixture was poured into cold water (2 L) and extracted with ethyl acetate (2×500 ml). Combined organic layer was washed with water, sat. NaHCO₃ and brine, dried over Na₂SO₄, concentrated and purified by column chromatography over silica gel using 20% ethyl acetate in pet ether as eluent to yield the title compound (22 g) as yellow solid.

### Step E 5-[4-(benzyloxy)phenyl]-1-(2,4-dichlorophenyl)-4-methyl-N-(5-methylpyridin-2-yl)-1H-pyrazole-3-carboxamide

5-Methylpyridin-2-amine (1.08 g, 10.0 mmol) was suspended in anhydrous toluene (10 mL) under an atmosphere of argon. Upon cooling to 0° C. trimethylaluminium (5.0 mL, 2.0 M in toluene, 10 mmol) was added dropwise at such a rate as to keep the evolution of methane under control. The obtained mixture was stirred at 0° C. for 30 min and then at ambient temperature for additional two hours before use. It was assumed that the formation of the desired aluminium amide was quantitative and the concentration thereof was thus calculated to be ca 0.67 M [c=10/(10+5.0)]. At that point ethyl 5-[4-(benzyloxy)phenyl]-1-(2,4-dichlorophenyl)-4-methyl-1H-pyrazole-3-carboxylate (1.00 g, 2.08 mmol) was added in one portion to the prepared aluminium amide and the resulting mixture was heated at 50° C. overnight. Next morning, as judged by LC/MS, unreacted starting material still remained. Therefore the reaction mixture was heated at 80° C. for three additional hours in order to drive the reaction to completion. Upon cooling to 0° C. the reaction was quenched by the dropwise addition of HCl (aq., 2 M) until further addition no longer resulted in gas evolution. At that point the ice bath was removed and the mixture was allowed to stir at ambient temperature for an additional hour. The obtained mixture was transferred to a separation funnel with the aid of CH₂Cl₂ (100 mL). H₂O (100 mL) was added and the pH of the aqueous phase was adjusted to 9-10. The organic phase was separated and the aqueous phase was extracted further with CH₂Cl₂ (5×30 mL). The collected organic phases (emulsion-like, brine was of no use) were dried over large amounts of MgSO₄. Upon evaporation of the solvents the obtained residue was purified by column chromatography (silica gel, EtOAc-CH₂Cl₂, 0-4%) to yield the desired 5-[4-(benzyloxy)phenyl]-1-(2,4-dichlorophenyl)-4-methyl-N-(5-methylpyridin-2-yl)-1H-pyrazole-3-carboxamide (1.020 g, 1.88 mmol, 90%) as a pinkish solid.

¹H NMR (500 MHz, CDCl₃) δ 9.40 (s, 1H), 8.28 (d, 1H, J=8.4 Hz), 8.14 (s, 1H), 7.56 (d, 1H, J=8.5 Hz), 7.48-7.26 (m, 8H), 7.09 (d, 2H, J=8.9 Hz), 6.94 (d, 2H, J=8.8 Hz), 5.06 (s, 2H), 2.44 (s, 3H), 2.32 (s, 3H).

### Step F 1-(2,4-dichlorophenyl)-5-(4-hydroxyphenyl)-4-methyl-N-(5-methylpyridin-2-yl)-1H-pyrazole-3-carboxamide

The 5-[4-(benzyloxy)phenyl]-1-(2,4-dichlorophenyl)-4-methyl-N-(5-methylpyridin-2-yl)-1H-pyrazole-3-carboxamide (0.100 g, 0.184 mmol) was put in a round flask and a solution of HBr in acetic acid (ca. 4.1 M, 1.8 mL) was added. After stirring at ambient temperature for four hours the reaction mixture was poured onto ice (20 g). Upon melting the pH was adjusted to ca. 7 by the addition of solid Na₂CO₃. The mixture was transferred to a separation funnel with the aid of CH₂Cl₂ (30 mL). The organic phase was separated and the aqueous phase was extracted further with CH₂Cl₂ (5×10 mL). The collected organic phases were dried over MgSO₄. Evaporation of the solvents yielded the crude 1-(2,4-dichlorophenyl)-5-(4-hydroxyphenyl)-4-methyl-N-(5-methylpyridin-2-yl)-1H-pyrazole-3-carboxamide (83 mg, 0.18 mmol, 99%) as a white solid of sufficient purity for the next step. ¹H NMR (500 MHz, MeOD-THF-d₈ (1:1)) δ 8.17 (d, 1H, J=8.4 Hz), 8.05 (s, 1H), 7.58 (d, 1H, J=9.0 Hz), 7.54 (s, 1H), 7.45 (d, 1H, J=8.4 Hz), 7.39 (d, 1H, J=8.5 Hz), 6.97 (d, 2H, J=8.7 Hz), 6.67 (d, 2H, J=8.7 Hz), 2.30 (s, 3H), 2.24 (s, 3H).

### Step G 4-(1-(2,4-dichlorophenyl)-4-methyl-3-{[(5-methylpyridin-2-yl)amino]carbonyl}-1H-pyrazol-5-yl)phenyl 3-methylbutane-1-sulfonate

The crude 1-(2,4-dichlorophenyl)-5-(4-hydroxyphenyl)-4-methyl-N-(5-methylpyridin-2-yl)-1H-pyrazole-3-carboxamide (0.076 g, 0.17 mmol) was suspended in anhydrous CH₂Cl₂ (1.7 mL) under an atmosphere of Ar and triethylamine (35 μL, 25 mg, 0.25 mmol) was added in one portion. Upon cooling to 0° C. 3-methylbutane-1-sulfonyl chloride (34 mg, 0.20 mmol) was added dropwise during one min. When the addition had been completed the ice bath was removed and the reaction mixture was allowed to reach ambient temperature. After stirring overnight the mixture was transferred to a separation funnel with the aid of CH₂Cl₂ (30 mL). H₂O (30 mL) was added. The organic phase was separated and the aqueous phase was extracted further with CH₂Cl₂ (3×10 mL). The collected organic phases were dried over MgSO₄. Upon evaporation of the solvents the obtained residue was purified by column chromatography (silica gel, EtOAc-CH₂Cl₂, 0-10%) to yield the desired 4-(1-(2,4-dichlorophenyl)-4-methyl-3-{[(5-methylpyridin-2-yl)amino]carbonyl}-1H-pyrazol-5-yl)phenyl 3-methylbutane-1-sulfonate (69 mg, 0.12 mmol, 70%) as a colourless, viscous oil. ¹H NMR (500 MHz, CDCl₃) δ 9.38 (s, 1H), 8.27 (d, 1H, J=8.4 Hz), 8.14 (s, 1H), 7.56 (d, 1H, J=8.4 Hz), 7.45 (s, 1H), 7.34 (d, 1H, J=8.4 Hz), 7.31 (d, 1H, J=8.3 Hz), 7.27 (d, 2H, J=8.5 Hz), 7.21 (d, 2H, J=8.8 Hz), 3.27 (m, 2H), 2.45 (s, 3H), 2.32 (s, 3H), 1.88 (m, 2H), 1.77 (m, 1H), 0.98 (d, 6H, J=6.6 Hz). HRMS Calcd for [C₂₈H₂₈Cl₂N₄O₄S+H]⁺: 587.1287. Found: 587.1332.

### Example 10

### 3,3,3-Trifluoro-propane-1-sulfonic acid 4-[2-(2,4-dichlorophenyl)-4-methyl-5-(5-methyl-pyridin-2-ylcarbamoyl)-2H-pyrazol-3-yl]phenyl ester

The crude 1-(2,4-dichlorophenyl)-5-(4-hydroxyphenyl)-4-methyl-N-(5-methylpyridin-2-yl)-1H-pyrazole-3-carboxamide, Ex. 9, Step F (0.100 g, 0.221 mmol) was suspended in anhydrous CH₂Cl₂ (2.2 mL) under an atmosphere of Ar and triethylamine (65 μL, 47 mg, 0.47 mmol) was added in one portion. Upon cooling to 0° C. 3,3,3-trifluoropropane-1-sulfonyl chloride (65 mg, 0.33 mmol) was added dropwise during ca. 5 min. When the addition had been completed the ice bath was removed and the reaction mixture was allowed to reach ambient temperature. After stirring overnight the mixture was transferred to a separation funnel with the aid of CH₂Cl₂ (30 mL). H₂O (30 mL) was added. The organic phase was separated and the aqueous phase was extracted further with CH₂Cl₂ (2×10 mL). The collected organic phases were dried over MgSO₄. Upon evaporation of the solvents the obtained residue was purified by column chromatography (silica gel, EtOAc-CH₂Cl₂, 0-5%) to yield the 4-(1-(2,4-dichlorophenyl)-4-methyl-3-{[(5-methylpyridin-2-yl)amino]carbonyl}-1H-pyrazol-5-yl)phenyl 3,3,3-trifluoropropane-1-sulfonate (88 mg, 0.14 mmol, 65%) as a colourless, viscous oil. ¹H NMR (500 MHz, CDCl₃) δ 9.41 (s, 1H), 8.28 (d, 1H, J=8.2 Hz), 8.14 (s, 1H), 7.58 (d, 1H, J=8.5 Hz), 7.46 (s, 1H), 7.38-7.30 (m, 2H), 7.30-7.20 (m, 4H), 3.58-3.44 (m, 2H), 2.90-2.74 (m, 2H), 2.46 (s, 3H), 2.33 (s, 3H). HRMS Calcd for [C₂₆H₂₁Cl₂F₃N₄O₄S+H]⁺: 613.0691. Found: 613.0724.

### Example 11

### (−)-4-[1-(2,4-dichlorophenyl)-3-({[cis-2-hydroxycyclohexyl]amino}carbonyl)-4-methyl-1H-pyrazol-5-yl]phenyl 3,3,3-trifluoropropane-1-sulfonate

The enantiomer was purified by preparatory HPLC (Chiralpak AD column, heptane:IPA 80:20) from Ex. 3 step G to give an almost white powder (661 mg, ee=98.6%) [α]_{D}²⁰=−7.5 (c 1.07, acetonitrile). ¹H NMR (399.964 MHz) δ 7.39 (s, 1H), 7.30-7.15 (m, 7H), 4.17-4.07 (m, 1H), 4.05-3.99 (m, 1H), 3.50-3.43 (m, 2H), 2.84-2.70 (m, 2H), 2.35 (s, 3H), 2.05-1.90 (br, 1H), 1.80-1.50 (m, 6H), 1.50-1.35 (m, 2H). HRMS Calcd for [C₂₆H₂₆Cl₂F₃N₃O₅S+H]⁺: 620.100. Found: 620.097.

### Example 12

### (+)-4-[1-(2,4-dichlorophenyl)-3-({[cis-2-hydroxycyclohexyl]amino}carbonyl)-4-methyl-1H-pyrazol-5-yl]phenyl 3,3,3-trifluoropropane-1-sulfonate

The enantiomer was purified by preparatory HPLC (Chiralpak AD column, heptane:IPA 80:20) from Ex. 3 step G to give an almost white powder (634 mg, ee=99.8%).

[α]_{D}²⁰=+7.3 (c 1.32, acetonitrile). ¹H NMR (399.964 MHz) δ 7.39 (s, 1H), 7.30-7.15 (m, 7H), 4.17-4.07 (m, 1H), 4.05-3.99 (m, 1H), 3.50-3.43 (m, 2H), 2.84-2.70 (m, 2H), 2.35 (s, 3H), 2.05-1.90 (br, 1H), 1.80-1.50 (m, 6H), 1.50-1.35 (m, 2H). HRMS Calcd for [C₂₆H₂₆Cl₂F₃N₃O₅S+H]⁺: 620.100. Found: 620.099.

### Example 13

### 4-[1-(2,4-dichlorophenyl)-3-({[3-(dimethylamino)cyclohexyl]amino}carbonyl-4-methyl-1H-pyrazol-5-yl]phenyl 3,3,3-trifluoropropane-1-sulfonate

### Step A: N-(3-aminocyclohexyl)-5-[4-(benzyloxy)phenyl]-1-(2,4-dichlorophenyl)-4-methyl-1H-pyrazole-3-carboxamide

Oxalyl chloride (2 ml) was added to 5-[4-(benzyloxy)phenyl]-1-(2,4-dichlorophenyl)-4-methyl-1H-pyrazole-3-carboxylic acid, prepare as in Ex. 3, Step D (400 mg, 0.88 mmol) in DCM (10 ml). One drop of DMF was added and the reaction continued at room temperature for 50 minutes. The solvent and excess oxalyl chloride was evaporated and the mixture dissolved in DCM (100 ml) and added dropwise to 1,3-cyclohexanediamine (2.01 g, 17.65 mmol) in DCM/K₂CO₃(10%, aq) (1:1, 40 ml). The phases were separated and the organic phase washed with water and dried over MgSO₄ (538 mg, crude). MS m/z 549, 551, 553 (M+H)⁺.

### Step B: 5-[4-(benzyloxy)phenyl]-1-(2,4-dichlorophenyl)-N-[3-(dimethylamino)cyclohexyl]-4-methyl-1H-pyrazole-3-carboxamide

N-(3-aminocyclohexyl)-5-[4-(benzyloxy)phenyl]-1-(2,4-dichlorophenyl)-4-methyl-1H-pyrazole-3-carboxamide (231 mg, crude) was dissolved/suspended in 5 ml acetonitrile. Formaldehyde (160 μl, 36%, aq) and NaBH₃CN (43 mg, 2.03 mmol) were added and the reaction continued at room temperature for 1 h. 3M NaOH (aq) was added until pH=10 and the mixture stirred for 1 h. DCM and water were added. The phases separated and the organic phase washed with water and dried over MgSO₄. (240 mg, crude).

MS m/z 577, 579, 581 (M+H)⁺.

### Step C: 1-(2,4-dichlorophenyl)-N-[3-(dimethylamino)cyclohexyl]-5-(4-hydroxyphenyl)-4-methyl-1H-pyrazole-3-carboxamide

Dimethyl sulfide (305 μl, 4.16 mmol) and boron trifluoride diethyl etherate (527 μl, 4.16 mmol) were added to 5-[4-(benzyloxy)phenyl]-1-(2,4-dichlorophenyl)-N-[3-(dimethylamino)cyclohexyl]-4-methyl-1H-pyrazole-3-carboxamide (240 mg, crude) in DCM (5 ml). The reaction was continued at room temperature for 91 hours. Water was added and the phases separated. The water phase was extracted with ethylacetate. The organic phase was washed with water and dried over MgSO₄ (227 mg, crude).

MS m/z 487, 489, 491 (M+H)⁺.

### Step D: 4-[1-(2,4-dichlorophenyl)-3-({[3-(dimethylamino)cyclohexyl]amino}carbonyl)-4-methyl-1H-pyrazol-5-yl]phenyl 3,3,3-trifluoropropane-1-sulfonate

A solution of 3,3,3-trifluoropropane-1-sulfonyl chloride (166 mg, 0.85 mmol) in DCM (2 ml) was added to a mixture of 1-(2,4-dichlorophenyl)-N-[3-(dimethylamino)cyclohexyl]-5-(4-hydroxyphenyl)-4-methyl-1H-pyrazole-3-carboxamide (227 mg, crude) and TEA (97 μl, 0.70 mmol) in DCM (6 ml) at −78° C., under N₂(g). The reaction was continued at −78° C. for 1 hour. Water was added and the phases were separated. The organic phase was washed with water and dried over MgSO₄. (241 mg, crude). MS m/z 647, 649, 651 (M+H)⁺.

### Example 14

### 4-[1-(2,4-dichlorophenyl)-3-({[trans-3-(dimethylamino)cyclohexyl]amino}carbonyl)-4-methyl-1H-pyrazol-5-yl]phenyl 3,33-trifluoropropane-1-sulfonate

The trans racemic mixture was separated from Ex. 13 Step D by prep LC (kromasil C8 column, ammonium acetate (aq, 0.1 M):acetonitrile, product came at 95% acetonitrile) to give a white powder after freeze drying. (26 mg, 10% yield for 4 steps). ¹H NMR (399.964 MHz) δ 7.45-7.12 (m, 7H), 6.96 (d, 1H), 4.48-4.38 (br, 1H), 3.52-3.40 (m, 2H), 2.84-2.68 (m, 2H), 2.50-2.40 (br, 1H), 2.35 (s, 3H), 2.30 (s, 6H), 2.00-1.35 (m, 8H). HRMS Calcd for [C₂₈H₃₁Cl₂F₃N₄O₄S+H]⁺: 647.147. Found: 647.148.

### Example 15

### 4-[1-(2,4-dichlorophenyl)-3-({[cis-3-(dimethylamino)cyclohexyl]amino}carbonyl)-4-methyl-1H-pyrazol-5-yl]phenyl 3,33-trifluoropropane-1-sulfonate

The cis racemic mixture was separated from Ex 13 Step D by prep LC (kromasil C8 column, ammonium acetate (aq, 0.1 M):acetonitrile, product came at 100% acetonitrile) to give a white powder after freeze drying. (13 mg, 5.3% yield for 4 steps). ¹H NMR (399.964 MHz) δ 7.43-7.15 (m, 7H), 7.02-6.90 (br, 1H), 4.05-3.91 (m, 1H), 3.50-3.41 (m, 2H), 2.85-2.70 (m, 2H), 2.36 (s, 3H), 2.35-2.28 (m, 1H), 2.26 (s, 6H), 2.25-1.80 (m, 4H), 1.45-1.05 (m, 4H). HRMS Calcd for [C₂₈H₃₁Cl₂F₃N₄O₄S+H]⁺: 647.147. Found: 647.148.

### Example 16

### 4-[3-({[cis-3-aminocyclohexyl]amino}carbonyl-1-(2,4-dichlorophenyl-4-methyl-1H-pyrazol-5-yl]phenyl 3,3,3-trifluoropropane-1-sulfonate hydrochloride

### Step A: N-(3-aminocyclohexyl)-5-[4-(benzyloxy)phenyl]-1-(2,4-dichlorophenyl)-4-methyl-1H-pyrazole-3-carboxamide

Oxalyl chloride (1 ml) was added to 5-[4-(benzyloxy)phenyl]-1-(2,4-dichlorophenyl)-4-methyl-1H-pyrazole-3-carboxylic acid, prepared as in Ex. 3, Step D (200 mg, 0.44 mmol) in DCM (5 ml). One drop of DMF was added and the reaction continued at room temperature for 1 hour. The solvent and excess oxalyl chloride was evaporated and the mixture dissolved in DCM (50 ml) and added dropwise to 1,3-cyclohexanediamine (1.01 g, 8.82 mmol) in DCM/K₂CO₃(10%, aq) (1:1, 20 ml). The phases were separated and the organic phase washed with water and dried over MgSO₄ (259 mg, crude).

MS m/z 549, 551, 553 (M+H)⁺.

### Step B: N-(3-aminocyclohexyl)-1-(2,4-dichlorophenyl)-5-(4-hydroxyphenyl)-4-methyl-1H-pyrazole-3-carboxamide

Dimethyl sulfide (346 μl, 4.71 mmol) and boron trifluoride diethyl etherate (597 μl, 4.71 mmol) were added to N-(3-aminocyclohexyl)-5-[4-(benzyloxy)phenyl]-1-(2,4-dichlorophenyl)-4-methyl-1H-pyrazole-3-carboxamide (259 mg, crude) in DCM (5 ml). The reaction was continued at room temperature for 70 hours. Water was added and the phases separated. The water phase was extracted with ethylacetate. The organic phase was washed with water and dried over MgSO₄ (207 mg, crude).

MS m/z 459, 461, 463 (M+H)⁺.

### Step C: N-(3-aminocyclohexyl)-5-(4-{[tert-butyl(dimethyl)silyl]oxy}phenyl)-1-(2,4-dichlorophenyl)-4-methyl-1H-pyrazole-3-carboxamide

A solution of tert-butyl(chloro)dimethylsilane (538 mg, 3.57 mmol) in DCM (2 ml) was added to a suspension of N-(3-aminocyclohexyl)-1-(2,4-dichlorophenyl)-5-(4-hydroxyphenyl)-4-methyl-1H-pyrazole-3-carboxamide (207 mg, crude) in DCM (5 ml) and TEA (754 μl, 5.41 mmol). The reaction was continued at room temperature for 24 hours. Water was added, the phases separated and the organic phase washed with water and dried over MgSO₄ (454 mg, crude). MS m/z 573, 575, 577 (M+H)⁺.

### Step D: tert-butyl [3-({[5-(4-{[tert-butyl(dimethyl)silyl]oxy}phenyl)-1-(2,4-dichlorophenyl)-4-methyl-1H-pyrazol-3-yl]carbonyl}amino)cyclohexyl]carbamate

A solution of di-tert-butyl dicarbonate (163 mg, 0.75 mmol) in THF (1 ml) was added to N-(3-aminocyclohexyl)-5-(4-{[tert-butyl(dimethyl)silyl]oxy}phenyl)-1-(2,4-dichlorophenyl)-4-methyl-1H-pyrazole-3-carboxamide (454 mg, crude) in THF (2 ml). The reaction was continued at room temperature for 2.5 hours. The solvent was evaporated at reduced pressure. Water and DCM were added. The phases were separated and the organic phase washed with NaOH (0.2 M, aq) and water and dried over MgSO₄. The product was further purified by flash chromatography (SiO₂, heptane/ethyl acetate, product came at 100% ethyl acetate (149 mg, 50% yield for 4 steps). MS m/z 673, 675, 677 (M+H)⁺.

### Step E: tert-butyl [3-({[1-(2,4-dichlorophenyl)-5-(4-hydroxyphenyl)-4-methyl-H-pyrazol-3-yl]carbonyl}amino)cyclohexyl]carbamate

TBAF (1M in THF, 3.2 ml) and acetic acid (142 μl, 2.48 mmol) were added to tert-butyl [3-({[5-(4-{[tert-butyl(dimethyl)silyl]oxy}phenyl)-1-(2,4-dichlorophenyl)-4-methyl-1H-pyrazol-3-yl]carbonyl}amino)cyclohexyl]carbamate (143 mg, 0.21 mmol) in THF (5 ml) under N₂(g). The reaction was continued at room temperature under N₂(g) for 1 hour. Silica gel was added. The mixture filtered through a small silica plug. The plug was washed with ethyl acetate. The solution was washed with water and dried over MgSO₄ (123 mg, crude). MS m/z 559, 561, 563 (M+H)⁺.

### Step F: 4-[3-[({3-[(tert-butoxycarbonyl)amino]cyclohexyl}amino)carbonyl]-1-(2,4-dichlorophenyl)-4-methyl-1H-pyrazol-5-yl]phenyl 3,3,3-trifluoropropane-1-sulfonate

A solution of 3,3,3-trifluoropropane-1-sulfonyl chloride (100 mg, 0.51 mmol) in DCM (2 ml) was added to a mixture of tert-butyl [3-({[1-(2,4-dichlorophenyl)-5-(4-hydroxyphenyl)-4-methyl-1H-pyrazol-3-yl]carbonyl}amino)cyclohexyl]carbamate (123 mg, crude) and TEA (50 μl, 0.36 mmol) in DCM (3 ml) at −78° C., under N₂(g). The reaction was continued at −78° C. for 50 minutes. Water was added and the phases were separated. The organic phase was washed with water and dried over MgSO₄. (177 mg, crude). MS m/z 719, 721, 723 (M+H)⁺.

### Step G 4-[3-[({cis-3-[(tert-butoxycarbonyl)amino]cyclohexyl}amino)carbonyl]-1-(2,4-dichlorophenyl)-4-methyl-1H-pyrazol-5-yl]phenyl 3,3,3-trifluoropropane-1-sulfonate

The product was separated from its isomer by prep LC (kromasil C8 column, ammonium acetate (aq, 0.1 M):acetonitrile, product came at 100% acetonitrile) to give a white powder after freeze drying. (60 mg, 39% yield for 2 steps). ¹H NMR (399.964 MHz) δ 7.42-7.14 (m, 7H), 6.77 (d, 1H), 4.40 (d, 1H), 4.04-3.91 (m, 1H), 3.60-3.42 (br, 1H), 3.50-3.42 (m, 2H), 2.84-2.68 (m, 2H), 2.34 (s, 3H), 2.35-1.65 (m, 4H), 1.40 (s, 9H), 1.45-0.90 (m, 4H). MS m/z 719, 721, 723 (M+H)⁺.

### Step H 4-[3-({[cis-3-aminocyclohexyl]amino}carbonyl)-1-(2,4-dichlorophenyl)-4-methyl-1H-pyrazol-5-yl]phenyl 3,3,3-trifluoropropane-1-sulfonate hydrochloride

HCl (4M in dioxane, 5 ml) was added to 4-[3-[({cis-3-[(tert-butoxycarbonyl)-amino]cyclohexyl}amino)carbonyl]-1-(2,4-dichlorophenyl)-4-methyl-1H-pyrazol-5-yl]phenyl 3,3,3-trifluoropropane-1-sulfonate (60 mg, 0.08 mmol) and reacted at room temperature for 45 minutes. The solvent was evaporated and the compound freeze-dried (54 mg, 99%). ¹H NMR 499.962 MHz) δ 7.61-7.32 (m, 7H), 4.04-3.94 (m, 1H), 3.75-3.70 (m, 2H), 3.28-3.20 (m, 1H), 2.94-2.82 (m, 2H), 2.34 (s, 3H), 2.38-2.30 (br, 1H), 2.10-1-94 (m, 3H), 1.60-1.30 (m, 4H). HRMS Calcd for [C₂₆H₂₇Cl₂F₃N₄O₄S+H]⁺: 619.116. Found: 619.117.

### Example 17

### 4-[3-({[trans-3-aminocyclohexyl]amino}carbonyl)-1-(2,4-dichlorophenyl)-4-methyl-1H-pyrazol-5-yl]phenyl 3,3,3-trifluoropropane-1-sulfonate hydrochloride

### Step A: 4-[3-[({trans-3-[(tert-butoxycarbonyl)amino]cyclohexyl}amino)carbonyl]-1-(2,4-dichlorophenyl)-4-methyl-1H-pyrazol-5-yl]phenyl 3,3,3-trifluoropropane-1-sulfonate

The trans racemic mixture was separated from 4-[3-[({3-[(tert-butoxycarbonyl)amino]-cyclohexyl}amino)carbonyl]-1-(2,4-dichlorophenyl)-4-methyl-1H-pyrazol-5-yl]phenyl 3,3,3-trifluoropropane-1-sulfonate, Ex. 16, Step F by prep LC (kromasil C8 column, ammonium acetate (aq, 0.1 M):acetonitrile, product came at 100% acetonitrile) to give a white powder after freeze drying. (31 mg, 20% yield for 2 steps).

¹H NMR (399.964 MHz) δ 7.42-7.14 (m, 7H), 6.94 (d, 1H), 4.70-4.62 (br, 1H), 4.30-3.20 (m, 1H), 3.90-3.80 (br, 1H), 3.50-3.42 (m, 2H), 2.84-2.68 (m, 2H), 2.35 (s, 3H), 1.85-1.40 (m, 8H), 1.40 (s, 9H). MS m/z 719, 721, 723 (M+H)⁺.

### Step B 4-[3-({[trans-3-aminocyclohexyl]amino}carbonyl)-1-(2,4-dichlorophenyl)-4-methyl-1H-pyrazol-5-yl]phenyl 3,3,3-trifluoropropane-1-sulfonate hydrochloride

HCl (4M in dioxane, 3 ml) was added to 4-[3-[({trans-3-[(tert-butoxycarbonyl)-amino]cyclohexyl}amino)carbonyl]-1-(2,4-dichlorophenyl)-4-methyl-1H-pyrazol-5-yl]phenyl 3,3,3-trifluoropropane-1-sulfonate (31 mg, 0.04 mmol) and reacted at room temperature for 1 hour. The solvent was evaporated and the compound freezedried (28 mg, 99%). ¹H NMR (499.962 MHz) δ 7.62-7.33 (m, 7H), 4.38-4.33 (m, 1H), 3.75-3.70 (m, 2H), 3.56-3.48 (m, 1H), 2.94-2.82 (m, 2H), 2.34 (s, 3H), 2.22-1.55 (m, 8H).

HRMS Calcd for [C₂₆H₂₇Cl₂F₃N₄O₄S+H]⁺: 619.116. Found: 619.117.

### Example 18

### 1-(2,4-dichlorophenyl)-4-methyl-N-piperidin-1-yl-5-[4-(3,3,3-trifluoropropoxy)phenyl]-1H-pyrazole-3-carboxamide

### Step A Ethyl 1-(2,4-dichlorophenyl)-5-(4-hydroxyphenyl)-4-methyl-1H-pyrazole-3-carboxylate

Ethyl 5-[4-(benzyloxy)phenyl]-1-(2,4-dichlorophenyl)-4-methyl-1H-pyrazole-3-carboxylate, Ex 9, Step D (4.82 g, 10 mmol) was dissolved in 80 ml HBr (33% in acetic acid) and stirred overnight at room temperature with exclusion of light. The solvents were evaporated and the residue coevaporated twice with ethanol. The residue was dissolved in EtOAc and washed with water basified with triethylamine and then brine. The organic layer was dried over Na₂SO₄ and evaporated to give ethyl 1-(2,4-dichlorophenyl)-5-(4-hydroxyphenyl)-4-methyl-1H-pyrazole-3-carboxylate (4.54 g) as a brown, viscous oil of sufficient purity for the next step. ¹H NMR (500 MHz, CDCl₃) δ 7.45-7.23 (m, 3H), 6.98 (d, J=8.7 Hz, 2H), 6.80 (d, J=8.7 Hz, 2H), 4.43 (q, J=7.1 Hz, 2H), 2.33 (s, 3H), 1.40 (t, J=7.1 Hz, 3H)

### Step B Ethyl 1-(2,4-dichlorophenyl)-4-methyl-5-[4-(3,3,3-trifluoropropoxy)phenyl]-1H-pyrazole-3-carboxylate

Ethyl 1-(2,4-dichlorophenyl)-5-(4-hydroxyphenyl)-4-methyl-1H-pyrazole-3-carboxylate (1.51 g, 3.87 mmol), 3,3,3-trifluoro-1-propanol (2.21 g, 19.4 mmol) and triphenylphosphine (5.08 g, 19.4 mmol) were dissolved in anhydrous THF (20 ml). Then DEAD (3.2 ml of a ca. 40% solution in toluene, d=0.95, 7.76 mmol) was added. The resulting mixture warms and was stirred at room temperature for 20 h, then an additional portion of DEAD (3.2 ml of a ca. 40% solution in toluene, d=0.95, 7.76 mmol) was added and stirring continued for 7 h, then again DEAD (1.6 ml of a ca. 40% solution in toluene, d=0.95, 3.88 mmol) was added and stirring continued for 16 h. The solvents were evaporated, the residue dissolved in 20 ml EtOAc and 80 ml of hexanes were added. Precipitation occurs. The resulting mixture was sonicated for ca. 5 min, the solid was filtered off and washed with hexanes/EtOAc 4:1. The combined filtrates were evaporated and the residue purified by column chromatography (silica gel, hexanes/EtOAc, 10-20%) to yield ethyl 1-(2,4-dichlorophenyl)-4-methyl-5-[4-(3,3,3-trifluoropropoxy)phenyl]-1H-pyrazole-3-carboxylate (1.81 g, 3.34 mmol, 86%) as a yellowish foam which contains ca. 10% of diethyl hydrazine-1,2-dicarboxylate, which does not interfere with the next transformation. ¹H NMR (400 MHz, CDCl₃) δ 7.35-7.22 (m, 3H), 7.00 (d, J=8.7 Hz, 2H), 6.81 (d, J=8.7 Hz, 2H), 4.43 (q, J=7.1 Hz, 2H), 4.18-4.13 (m, 2H), 2.65-2.55 (m, 2H), 2.30 (s, 3H), 1.40 (t, J=7.1 Hz, 3H)

### Step C 1-(2,4-dichlorophenyl)-4-methyl-5-[4-(3,3,3-trifluoropropoxy)phenyl]-1H-pyrazole-3-carboxylic acid

Ethyl 1-(2,4-dichlorophenyl)-4-methyl-5-[4-(3,3,3-trifluoropropoxy)phenyl]-1H-pyrazole-3-carboxylate (700 mg, 1.29 mmol, ca. 90% pure) was dissolved in a mixture of 15 ml THF and 15 ml EtOH, then KOH (870 mg, 15.5 mmol) dissolved in 10 ml water was added and the resulting mixture stirred at 50° C. After 1 h the reaction mixture was cooled to room temperature and the solvents were evaporated. The residue was partitioned between EtOAc and 1N HCl and after phase separation the organic layer was washed with brine, dried over Na₂SO₄ and evaporated to give 1-(2,4-dichlorophenyl)-4-methyl-5-[4-(3,3,3-trifluoropropoxy)phenyl]-1H-pyrazole-3-carboxylic acid (640 mg, 1.28 mmol, 99%) as a yellowish foam which was sufficiently pure for the next step. ¹H NMR (400 MHz, CDCl₃) δ 7.39-7.23 (m, 3H), 7.05 (d, J=8.7 Hz, 2H), 6.82 (d, J=8.7 Hz, 2H), 4.18-4.13 (m, 2H), 2.65-2.55 (m, 2H), 2.30 (s, 3H)

### Step D 1-(2,4-dichlorophenyl)-4-methyl-5-[4-(3,3,3-trifluoropropoxy)phenyl]-1H-pyrazole-3-carbonyl chloride

1-(2,4-dichlorophenyl)-4-methyl-5-[4-(3,3,3-trifluoropropoxy)phenyl]-1H-pyrazole-3-carboxylic acid (640 mg, 1.28 mmol) was dissolved in 10 ml DCM, then oxalyl chloride (200 μl, 2.36 mmol) was added, followed by 10 μl DMF. The resulting mixture was stirred for 90 min at room temperature, then the solvents were evaporated and the residue dried in oil pump vacuum to give 1-(2,4-dichlorophenyl)-4-methyl-5-[4-(3,3,3-trifluoropropoxy)phenyl]-1H-pyrazole-3-carbonyl chloride (664 mg, 1.39 mmol, 99%) as a yellowish foam which was used without further purification in the next step.

### Step E General procedure for the synthesis of 1-(2,4-dichlorophenyl)-4-methyl-5-[4-(3,33-trifluoropropoxy)phenyl]-1H-pyrazole-3-carboxamides

To a mixture of amine or amine hydrochloride (0.3 mmol) and 10011 pyridine in 1 ml DCM was added crude 1-(2,4-dichlorophenyl)-4-methyl-5-[4-(3,3,3-trifluoropropoxy)phenyl]-1H-pyrazole-3-carbonyl chloride (96 mg, 0.2 mmol) in 1 ml DCM and the resulting mixture stirred at room temperature for 2 h 30 min. The reaction mixture was washed with 2 ml of sat. NaHCO₃ and after phase separation filtered through a phase separator. The solvents were evaporated and the residue purified by preparative HPLC eluting on a reverse-phase column with 5 to 100% acetonitrile in 0.1 M NH₄Ac. 1-(2,4-dichlorophenyl)-4-methyl-N-piperidin-1-yl-5-[4-(3,3,3-trifluoropropoxy)phenyl]-1H-pyrazole-3-carboxamide

Using piperidin-1-amine hydrochloride as amine component gave 1-(2,4-dichlorophenyl)-4-methyl-N-piperidin-1-yl-5-[4-(3,3,3-trifluoropropoxy)phenyl]-1H-pyrazole-3-carboxamide (36 mg, 65 μmol, 33%) as a colorless solid. ¹H NMR (500 MHz, CDCl₃) δ 7.65 (s, 1H), 7.44-7.27 (m, 3H), 7.06 (d, J=8.7 Hz, 2H), 6.84 (d, J=8.7 Hz, 2H), 4.21-4.17 (m, 2H), 2.90-2.86 (m, 4H), 2.67-2.59 (m, 2H), 2.38 (s, 3H), 1.80-1.75 (m, 4H), 1.47-1.43 (m, 2H). HRMS Calcd for [C₂₅H₂₅Cl₂F₃N₄O₂+H]⁺: 541.1385. Found: 541.1365.

### Example 19

### N-cyclohexyl-1-(2,4-dichlorophenyl)-4-methyl-5-[4-(3,33-trifluoropropoxy)phenyl]-1H-pyrazole-3-carboxamide

Using cyclohexylamine as amine component gave N-cyclohexyl-1-(2,4-dichlorophenyl)-4-methyl-5-[4-(3,3,3-trifluoropropoxy)phenyl]-1H-pyrazole-3-carboxamide (43 mg, 80 μmol, 40%) as a colorless solid.

¹H NMR (500 MHz, CDCl₃) δ 7.44-7.28 (m, 3H), 7.07 (d, J=8.7 Hz, 2H), 6.86-6.83 (m, 3H), 4.21-4.17 (m, 2H), 4.01-3.93 (m, 1H), 2.68-2.59 (m, 2H), 2.38 (s, 3H), 2.06-2.02 (m, 2H), 1.79-1.75 (m, 2H), 1.67-1.64 (m, 1H), 1.47-1.37 (m, 2H), 1.34-1.16 (m, 3H)

HRMS Calcd for [C₂₆H₂₆Cl₂F₃N₃O₂+H]⁺: 540.1432. Found: 540.1439.

### Example 20

### 1-(2,4-dichlorophenyl)-N-[(cis)-2-hydroxycyclohexyl]-4-methyl-5-[4-(3,3,3-trifluoropropoxy)phenyl]-1H-pyrazole-3-carboxamide

Using cis-2-aminocyclohexanol hydrochloride as amine component gave 1-(2,4-dichlorophenyl)-N-[(cis)-2-hydroxycyclohexyl]-4-methyl-5-[4-(3,3,3-trifluoropropoxy)phenyl]-1H-pyrazole-3-carboxamide (32 mg, 58 μmol, 29%) as a colorless solid. ¹H NMR (500 MHz, CDCl₃) δ 7.43-7.28 (m, 3H), 7.23 (d, J=8.2 Hz, 1H), 7.07 (d, J=8.7 Hz, 2H), 6.84 (d, J=8.7 Hz, 2H), 4.19-4.16 (m, 3H), 4.07-4.05 (m, 1H), 2.67-2.59 (m, 4H), 2.37 (s, 3H), 2.32 (s, 1H), 1.81-1.45 (m, 6H). HRMS Calcd for [C₂₆H₂₆Cl₂F₃N₃O₃+H]⁺: 556.1382. Found: 556.1398.

### Example 21

### 1-(2,4-dichlorophenyl)-N-(4,4-difluorocyclohexyl)-4-methyl-5-[4-(3,3,3-trifluoropropoxy)phenyl]-1H-pyrazole-3-carboxamide

Using 4,4-difluorocyclohexylamine as amine component gave 1-(2,4-dichlorophenyl)-N-(4,4-difluorocyclohexyl)-4-methyl-5-[4-(3,3,3-trifluoropropoxy)phenyl]-1H-pyrazole-3-carboxamide (54 mg, 94 μmol, 47%) as a colorless solid. ¹H NMR (500 MHz, CDCl₃) δ 7.49-7.28 (m, 3H), 7.07 (d, J=8.7 Hz, 2H), 6.88-6.84 (m, 3H), 4.20-4.17 (m, 2H), 4.12-4.10 (m, 1H), 2.68-2.59 (m, 2H), 2.38 (s, 3H), 2.13-2.10 (m, 4H), 1.98-1.86 (m, 2H), 1.71-1.64 (m, 2H). HRMS Calcd for [C₂₆H₂₄Cl₂F₅N₃O₂+H]⁺: 576.1244. Found: 576.1262.

### Example 22

### 1-(2,4-dichlorophenyl)-4-methyl-N-(5-methylpyridin-2-yl)-5-[4-(3,33-trifluorolpropoxy)phenyl]-1H-pyrazole-3-carboxamide

Using 2-amino-5-picoline as amine component gave 1-(2,4-dichlorophenyl)-4-methyl-N-(5-methylpyridin-2-yl)-5-[4-(3,3,3-trifluoropropoxy)phenyl]-1H-pyrazole-3-carboxamide (52 mg, 95 μmol, 47%) as a colorless solid. ¹H NMR (500 MHz, CDCl₃) δ 9.39 (s, 1H), 8.28 (d, J=8.3 Hz, 1H), 8.14 (s, 1H), 7.57-7.55 (m, 1H), 7.45-7.28 (m, 3H), 7.10 (d, J=8.7 Hz, 2H), 6.86 (d, J=8.7 Hz, 2H), 4.22-4.18 (m, 2H), 2.68-2.61 (m, 2H), 2.44 (s, 3H), 2.32 (s, 3H) HRMS Calcd for [C₂₆H₂₁Cl₂F₃N₄O₂+H]⁺: 549.1072. Found: 549.1074.

### Example 23

### 4-[1-(2-chlorophenyl)-3-{[(1S,2R)-2-hydroxycyclohexyl]carbamoyl}-4-methyl-1H-pyrazol-5-yl]phenyl 3,3,3-trifluoropropane-1-sulfonate and 4-[1-(2-chlorophenyl)-3-{[(1R,2S)-2-hydroxycyclohexyl]carbamoyl}-4-methyl-1H-pyrazol-5-yl]phenyl 3,3,3-trifluoropropane-1-sulfonate

### Step A: Lithium-1-[4-(benzyloxy)phenyl]-4-ethoxy-2-methyl-3,4-dioxobut-1-en-1-olate

Para-benzyloxypropiophenone (3.84 g, 15.98 mmol) in dry THF (30 ml) was added to a solution of lithium bis(trimethylsilyl)amide (17.6 ml, 1M in hexanes) in diethyl ether (100 ml) at −78° C., under N₂ (g). The reaction was continued at −78° C., under N₂ (g) for 1 hour. Ethyl oxalate (2.44 ml, 18.04 mmol) was added. The reaction was continued at room temperature for 19 hours. The mixture was filtered and the filtrate washed with THF/diethyl ether 1:5 and diethyl ether and evaporated under reduced pressure (3.66 g crude).

### Step B: Ethyl 5-[4-(benzyloxy)phenyl]-1-(2-chlorophenyl)-4-methyl-1H-pyrazole-3-carboxylate

Ethyl 3-[4-(benzyloxy)phenyl]-2-methyl-3-oxopropanoate lithium salt or lithium-1-[4-(benzyloxy)phenyl]-4-ethoxy-2-methyl-3,4-dioxobut-1-en-1-olate (3.66 g, crude) and (2-chlorophenyl)hydrazine hydrochloride (1.30 g, 7.26 mmol) were mixed in ethanol (50 ml) and reacted at room temperature for 16 hours. The solvent was evaporated and the mixture was suspended in acetic acid (40 ml). The temperature was increased to 100° C. and the reaction continued for 4 hours. The solvent was evaporated. Water and DCM were added. The phases separated and the organic phase washed with water and dried over MgSO₄. The product was purified further by flash chromatography (SiO₂, heptane/ethyl acetate, product came at 30% ethyl acetate) (771 mg, 10% for two steps).

¹H NMR (399.964 MHz) δ 7.50-7-25 (m, 9H), 7.10 (d, 2H), 6.90 (d, 2H), 5.01 (s, 2H), 4.46 (q, 2H), 2.36 (s, 3H), 1.43 (t, 3H). MS m/z 447, 449 (M+H)⁺.

### Step C: 5-[4-(benzyloxy)phenyl]-1-(2-chlorophenyl)-4-methyl-1H-pyrazole-3-carboxylic acid

Ethyl 5-[4-(benzyloxy)phenyl]-1-(2-chlorophenyl)-4-methyl-1H-pyrazole-3-carboxylate (771 mg, 1.73 mmol) and sodium hydroxide (2.93 g, 73.13 mmol) were reacted in water/ethanol (1:5, 60 ml) for 1 hour. The solvent was evaporated and the mixture suspended in water and neutralised with HCl (conc.). The product was collected by filtration, washed with water and dried at reduced pressure (647 mg, crude).

¹H NMR (399.964 MHz) δ 7.40-7.20 (m, 9H), 7.07 (d, 2H). 6.88 (d, 2H), 5.00 (s, 2H), 2.34 (s, 3H). MS m/z 419, 421 (M+H)⁺.

### Step D: 2,2,2-trichloroethyl 5-[4-(benzyloxy)phenyl]-1-(2-chlorophenyl)-4-methyl-1H-pyrazole-3-carboxylate

Oxalyl chloride (1.5 ml) and 1 drop of DMF were added to a mixture of 5-[4-(benzyloxy)phenyl]-1-(2-chlorophenyl)-4-methyl-1H-pyrazole-3-carboxylic acid (632 mg, crude) in DCM (15 ml). The reaction was continued at room temperature for 1 hour. The solvent and excess oxalyl chloride were evaporated. The acid chloride was dissolved in DCM (10 ml) and added to a mixture of 2,2,2-trichloroethanol (325 mg, 12.18 mmol) and DIPEA (350 μl, 2.01 mmol) in DCM (5 ml). DMAP (6 mg, 0.05 mmol) was added and the reaction continued at room temperature for 3 hours. Water was added. The phases were separated and the organic phase washed with water, NaOH (3M, aq), HCl (2M, aq) and water, and dried over MgSO₄ (807 mg, crude).

¹H NMR (399.964 MHz) δ 7.40-7.22 (m, 9H), 7.07 (d, 2H), 6.88 (d, 2H), 5.04 (s, 2H), 4.99 (s, 2H), 2.37 (s, 3H). MS m/z 549, 551, 553, 555 (M+H)⁺.

### Step E: 2,2,2-trichloroethyl 1-(2-chlorophenyl)-5-(4-hydroxyphenyl)-4-methyl-1H-pyrazole-3-carboxylate

2,2,2-Trichloroethyl 5-[4-(benzyloxy)phenyl]-1-(2-chlorophenyl)-4-methyl-1H-pyrazole-3-carboxylate (807 mg, crude) was dissolved in HBr in acetic acid (33%, 10 ml) and reacted at room temperature for 1 hour. Ethanol was added and the mixture stirred for 1 hour. The solvent was evaporated. Methanol was added, the mixture neutralised with NaHCO₃ (5%, aq) and the solvent evaporated. Water and DCM were added. The phases were separated and the organic phase washed with water and dried over MgSO₄ (627 mg, crude). ¹H NMR (399.964 MHz) δ 7.36-7.20 (m, 4H), 6.96 (d, 2H), 6.69 (d, 2H), 6.10-5.60 (br, 1H), 5.01 (s, 2H), 2.34 (s, 3H). MS m/z/z 459, 461, 463, 465 (M+H)⁺.

### Step F: 2,2,2-trichloroethyl 1-(2-chloro-phenyl)-4-methyl-5-(4-{[(3,3,3-trifluoropropyl)sulfonyl]oxy}phenyl)-1H-pyrazole-3-carboxylate

3,3,3-Trifluoropropane-1-sulfonyl chloride (350 mg, 1.78 mmol) in DCM (2 ml) was added to a mixture of 2,2,2-trichloroethyl 1-(2-chlorophenyl)-5-(4-hydroxyphenyl)-4-methyl-1H-pyrazole-3-carboxylate (595 mg, crude) and TEA (250 μl, 1.79 mmol) in DCM (10 ml) at −78° C., under N₂(g). The reaction was continued at −78° C. for 1 hour. Water was added, the phases were separated and the organic phase was washed with water and dried (865 mg, crude). ¹H NMR (399.964 MHz) δ 7.45-7.15 (m, 8H), 5.02 (s, 2H), 3.48-3.42 (m, 2H), 2.82-2.68 (m, 2H), 2.36 (s, 3H). MS m/z 619, 621, 623, 625 (M+H)⁺.

### Step G: 1-(2-chlorophenyl)-4-methyl-5-(4-{[(3,3,3-trifluoropropyl)sulfonyl]oxy}phenyl)-1H-pyrazole-3-carboxylic acid

Zinc dust (840 mg, 12.85 mmol) was added to a mixture of 2,2,2-trichloroethyl 1-(2-chlorophenyl)-4-methyl-5-(4-{[(3,3,3-trifluoropropyl)sulfonyl]oxy}phenyl)-1H-pyrazole-3-carboxylate (865 mg, crude) in acetic acid (10 ml). The reaction was continued at room temperature for 1 hour. DCM was added and the mixture was filtered through Celite 521. The solvent was evaporated and the mixture dissolved in DCM and washed with HCl (1M, aq) and water, and dried over MgSO₄. The mixture was dried further by co-evaporation with toluene (599 mg, crude). ¹H NMR (399.964 MHz) δ 7.13-6.80 (m, 8H), 3.20-3.10 (m, 2H), 2.54-2.38 (m, 2H), 2.06 (s, 3H). MS m/z 489, 491 (M+H)⁺.

### Step H: 4-[3-(chlorocarbonyl)-1-(2-chlorophenyl)-4-methyl-1H-pyrazol-5-yl]phenyl 3,3,3-trifluoropropane-1-sulfonate

Oxalyl chloride (1.5 ml) was added to 1-(2-chlorophenyl)-4-methyl-5-(4-{[(3,3,3-trifluoropropyl)sulfonyl]oxy}phenyl)-1H-pyrazole-3-carboxylic acid (599 mg, crude) in DCM (10 ml). One drop of DMF was added and the reaction continued at room temperature for 1.5 hours. The solvent and excess oxalyl chloride were evaporated at reduced pressure.

### Step I: 4-[1-(2-chlorophenyl)-3-{[(1S,2R)-2-hydroxycyclohexyl]carbamoyl}-4-methyl-1H-pyrazol-5-yl]phenyl 3,3,3-trifluoropropane-1-sulfonate and 4-[1-(2-chlorophenyl)-3-{[(1R,2S)-2-hydroxycyclohexyl]carbamoyl}-4-methyl-1H-pyrazol-5-yl]phenyl 3,3,3-trifluoropropane-1-sulfonate

4-[3-Chlorocarbonyl)-1-(2-chlorophenyl)-4-methyl-1H-pyrazol-5-yl]phenyl 3,3,3-trifluoropropane-1-sulfonate (207 mg, crude) suspended in DCM (3 ml) was added to cis-2-aminocyclohexanol hydrochloride (81 mg, 0.53 mmol) in DCM/K₂CO₃ (10%, aq) (1:1, 6 ml). The reaction was continued at room temperature for 1 hour. The phases were separated and the organic phase was washed with water and dried. The product was further purified by preparatory HPLC (kromasil C8 column, ammonium acetate (aq, 0.1 M):acetonitrile, product came at 96% acetonitrile) to give an almost white powder (135 mg, 43% yield for 7 steps). ¹H NMR (399.964 MHz) δ 7.32-7.07 (m, 9H), 4.08-3.97 (m, 1H), 3.97-3.89 (m, 1H), 3.40-3.32 (m, 2H), 2.74-2.58 (m, 2H), 2.28 (s, 3H), 1.70-1.25 (m, 9H). HRMS Calcd for [C₂₆H₂₇ClF₃N₃O₅S+H]⁺: 586.139. Found: 586.142.

### Example 24

### 4-[1-(2-chlorophenyl)-3-(cyclohexylcarbamoyl)-4-methyl-1H-pyrazol-5-yl]phenyl 3,3,3-trifluoropropane-1-sulfonate

4-[3-(chlorocarbonyl)-1-(2-chlorophenyl)-4-methyl-1H-pyrazol-5-yl]phenyl 3,3,3-trifluoropropane-1-sulfonate, from Ex. 23, Step H (207 mg, crude) suspended in DCM (3 ml) was added to cyclohexylamine (167 mg, 1.68 mmol) in DCM/K₂CO₃ (10%, aq) (1:1, 6 ml). The reaction was continued at room temperature for 1 hour. The phases were separated and the organic phase was washed with water and dried. The product was further purified by preparatory HPLC (kromasil C8 column, ammonium acetate (aq, 0.1 M):acetonitrile, product came at 100% acetonitrile) to give an almost white powder (155 mg, 51% yield for 7 steps). ¹H NMR (399.964 MHz) δ 7.42-7.15 (m, 8H), 6.83 (d, 1H), 4.00-3.86 (m, 1H), 3.48-3.40 (m, 2H), 2.82-2.68 (m, 2H), 2.38 (s, 3H), 2.05-1.10 (m, 10H).

HRMS Calcd for [C₂₆H₂₇ClF₃N₃O₄S+H]⁺: 570.144. Found: 570.146.

### Example 25

### 4-{1-(4-chloro-2-methylphenyl)-4-methyl-3-[(piperidin-1-ylamino)carbonyl]-1H-pyrazol-5-yl}phenyl 3,3,3-trifluoropropane-1-sulfonate

### Step A: Ethyl 3-[4-(benzyloxy)phenyl]-2-methyl-3-oxopropanoate lithium salt (lithium-1-[4-(benzyloxy)phenyl]-4-ethoxy-2-methyl-3,4-dioxobut-1-en-1-olate)

Para-benzyloxypropiophenone (1.92 g, 7.99 mmol) in dry THF (15 ml) was added to a solution of lithium bis(trimethylsilyl)amide (8.8 ml, 1M in hexanes) in diethyl ether (50 ml) at −78° C., under N₂ (g). The reaction was continued at −78° C., under N₂ (g) for 1 hour. Ethyl oxalate (1.22 ml, 9.02 mmol) was added. The reaction was continued at room temperature for 21 hours. The mixture was filtered and the filtrate washed with THF/diethyl ether 1:5 and diethyl ether and dried at reduced pressure (1.09 g crude).

### Step B: Ethyl 5-[4-(benzyloxy)phenyl]-1-(4-chloro-2-methylphenyl)-4-methyl-1H-pyrazole-3-carboxylate

Ethyl 3-[4-(benzyloxy)phenyl]-2-methyl-3-oxopropanoate lithium salt (lithium-1-[4-(benzyloxy)phenyl]-4-ethoxy-2-methyl-3,4-dioxobut-1-en-1-olate) (1.09 g, crude) and (4-chloro-2-methylphenyl)hydrazine hydrochloride (0.846 g, 4.38 mmol) were mixed in acetic acid (20 ml) and reacted at room temperature for 17 hours. The temperature was increased to 100° C. and the reaction continued for 5 hours. The product was purified by flash chromatography (SiO₂, heptane/ethyl acetate, product came at 20% ethyl acetate) (577 mg, 15% for two steps). ¹H NMR (399.964 MHz) δ 7.40-7-20 (m, 5H), 7.12 (s, 3H), 6.99 (d, 2H), 6.87 (d, 2H), 4.99 (s, 2H), 4.42 (q, 2H), 2.32 (s, 3H), 1.89 (s, 3H), 1.39 (t, 3H). MS m/z 461, 463 (M+H)⁺.

### Step C: 5-[4-(benzyloxy)phenyl]-1-(4-chloro-2-methylphenyl)-4-methyl-1H-pyrazole-3-carboxylic acid

Ethyl 5-[4-(benzyloxy)phenyl]-1-(4-chloro-2-methylphenyl)-4-methyl-1H-pyrazole-3-carboxylate (577 mg, 1.25 mmol) and sodium hydroxide (2.15 g, 53.85 mmol) were reacted in water/ethanol (1:5, 60 ml) for 1 hour. The solvent was evaporated and the mixture suspended in water and neutralised with HCl (conc.). The product was collected by filtration, washed with water and dried at reduced pressure (576 mg, crude).

¹H NMR (399.964 MHz) δ 7.36-7.22 (m, 5H), 6.96-6.84 (m, 3H), 6.80-6.68 (4H), 4.89 (s, 2H), 1.99 (s, 3H), 1.56 (s, 3H). MS tn/z 433, 435 (M+H)⁺.

### Step D: 2,2,2-trichloroethyl 5-[4-(benzyloxy)phenyl]-1-(4-chloro-2-methylphenyl)-4-methyl-1H-pyrazole-3-carboxylate

Oxalyl chloride (1.5 ml) and 1 drop of DMF were added to a mixture of 5-[4-(benzyloxy)phenyl]-1-(4-chloro-2-methylphenyl)-4-methyl-1H-pyrazole-3-carboxylic acid (576 mg, crude) in DCM (6 ml). The reaction was continued at room temperature for 1 hour. The solvent and excess oxalyl chloride were evaporated. The acid chloride was dissolved in DCM (3 ml) and added to a mixture of 2,2,2-trichloroethanol (140 μl, 1.46 mmol) and DIPEA (280 μl, 1.60 mmol) in DCM (3 ml). The reaction was continued at room temperature for 2 hours. Water was added. The phases were separated and the organic phase washed with water, NaOH (aq), HCl (aq) and water, and dried over MgSO₄ (662 mg, crude). ¹H NMR (399.964 MHz) δ 7.44-7.28 (m, 5H), 7.17-7.10 (m, 3H), 7.00 (d, 2H), 6.89 (d, 2H), 5.04 (s, 2H), 5.01 (s, 2H), 2.37 (s, 3H), 1.93 (s, 3H). MS m/z 563, 565, 567, 569 (M+H)⁺.

### Step E: 2,2,2-trichloroethyl 1-(4-chloro-2-methylphenyl)-5-(4-hydroxyphenyl)-4-methyl-1H-pyrazole-3-carboxylate

2,2,2-trichloroethyl 5-[4-(benzyloxy)phenyl]-1-(4-chloro-2-methylphenyl)-4-methyl-1H-pyrazole-3-carboxylate (662 mg, crude) was dissolved in HBr in acetic acid (33%, 15 ml) and reacted at room temperature for 1 hour. Ethanol was added and the mixture stirred for 1.5 hours. The solvent was evaporated. Methanol was added, the mixture neutralised with NaHCO₃ (5%, aq) and the solvent evaporated. Water and DCM were added. The phases separated and the organic phase washed with water and dried over MgSO₄ (543 mg, crude). ¹H NMR (399.964 MHz) δ 7.10-7.04 (m, 3H), 6.84 (d, 2H), 6.66 (d, 2H), 4.99 (s, 2H), 2.33 (s, 3H), 1.85 (s, 3H). MS m/z 473, 475, 477, 479 (M+H)⁺.

### Step F: 2,2,2-trichloroethyl 1-(4-chloro-2-methylphenyl)-4-methyl-5-(4-{[(3,3,3-trifluoropropyl)sulfonyl]oxy}phenyl-1H-pyrazole-3-carboxylate

3,3,3-Trifluoropropane-1-sulfonyl chloride (320 mg, 1.63 mmol) in DCM (2 ml) was added to a mixture of 2,2,2-trichloroethyl 1-(4-chloro-2-methylphenyl)-5-(4-hydroxyphenyl)-4-methyl-1H-pyrazole-3-carboxylate (543 mg, crude) and TEA (240 μl, 1.72 mmol) in DCM (15 ml) at −78° C., under N₂(g). The reaction was continued at −78° C. for 1 hour. Water was added, the phases were separated and the organic phase was washed with water and dried over MgSO₄ (707 mg, crude).

¹H NMR (399.964 MHz) δ 7.25-7.05 (m, 7H), 5.01 (s, 2H), 3.50-3.42 (m, 2H), 2.82-2.68 (m, 2H), 2.35 (s, 3H), 1.92 (s, 3H). MS m/z 633, 635, 637, 639 (M+H)⁺.

### Step G: 1-(4-chloro-2-methylphenyl)-4-methyl-5-(4-{[(3,3,3-trifluoropropyl)sulfonyl]oxy}phenyl)-1H-pyrazole-3-carboxylic acid

Zinc dust (729 mg, 11.15 mmol) was added to a mixture of 2,2,2-trichloroethyl 1-(4-chloro-2-methylphenyl)-4-methyl-5-(4-{[(3,3,3-trifluoropropyl)sulfonyl]oxy}phenyl)-1H-pyrazole-3-carboxylate (707 mg, crude) in acetic acid (10 ml). The reaction was continued at room temperature for 1.5 hours. DCM was added and the mixture was filtered through Celite 521. The solvent was evaporated and the mixture dissolved in DCM and washed with HCl (1M, aq) and water, and dried over MgSO₄. The mixture was dried further by coevaporation with toluene (498 mg, crude). ¹H NMR (399.964 MHz) δ 7.25-7.05 (m, 7H), 3.52-3.42 (m, 2H), 2.84-2.70 (m, 2H), 2.36 (s, 3H), 1.93 (s, 3H). MS m/z 503, 505 (M+H)⁺.

### Step H: 4-[3-(chlorocarbonyl)-1-(4-chloro-2-methylphenyl)-4-methyl-1H-pyrazol-5-yl]phenyl 3,3,3-trifluoropropane-1-sulfonate

Oxalyl chloride (1 ml) was added to 1-(4-chloro-2-methylphenyl)-4-methyl-5-(4-{[(3,3,3-trifluoropropyl)sulfonyl]oxy}phenyl)-1H-pyrazole-3-carboxylic acid (378 mg, crude) in DCM (20 ml). One drop of DMF was added and the reaction continued at room temperature for 50 minutes. The solvent and excess oxalyl chloride was evaporated at reduced pressure.

### Step I: 4-{1-(4-chloro-2-methylphenyl)-4-methyl-3-[(piperidin-1-ylamino)carbonyl]-1H-pyrazol-5-yl}phenyl 3,3,3-trifluoropropane-1-sulfonate

4-[3-(Chlorocarbonyl)-1-(4-chloro-2-methylphenyl)-4-methyl-1H-pyrazol-5-yl]phenyl 3,3,3-trifluoropropane-1-sulfonate (196 mg, crude) suspended in DCM (5 ml) was added to piperidin-1-amine hydrochloride (78 mg, 0.57 mmol) in DCM/K₂CO₃ (10%, aq) (1:1, 6 ml). The reaction was continued at room temperature for 1.5 hours. The phases were separated and the organic phase was washed with water and dried over MgSO₄. The product was further purified by preparatory HPLC (kromasil C8 column, ammonium acetate (aq, 0.1 M):acetonitrile, product came at 99% acetonitrile) to give an almost white powder (144 mg, 51% yield for 7 steps). ¹H NMR (399.964 MHz) δ 7.70-7.55 (br, 1H), 7.26-7.04 (m, 7H), 3.50-3.44 (m, 2H), 2.89-2.70 (m, 6H), 2.37 (s, 3H), 1.92 (s, 3H), 1.77-1.69 (m, 4H), 1.45-1.36 (m, 2H). HRMS Calcd for [C₂₆H₂₈ClF₃N₄O₄S+H]⁺: 585.155.

Found: 585.155.

### Example 26

### 4-[1-(4-chloro-2-methylphenyl)-3-({[(1S,2R)-2-hydroxycyclohexyl]amino}carbonyl)-4-methyl-1H-pyrazol-5-yl]phenyl 3,3,3-trifluoropropane-1-sulfonate and 4-[1-(4-chloro-2-methylphenyl)-3-({[(1R,2S)-2-hydroxycyclohexyl]amino}carbonyl)-4-methyl-1H-pyrazol-5-yl]phenyl 3,3,3-trifluoropropane-1-sulfonate

4-[3-(chlorocarbonyl)-1-(4-chloro-2-methylphenyl)-4-methyl-1H-pyrazol-5-yl]phenyl 3,3,3-trifluoropropane-1-sulfonate, from Ex. 25, Step H (196 mg, crude) suspended in DCM (5 ml) was added to cis-2-aminocyclohexanol hydrochloride (76.8 mg, 0.51 mmol) in DCM/K₂CO₃ (10%, aq) (1:1, 6 ml). The reaction was continued at room temperature for 2 hours. The phases were separated and the organic phase was washed with water and dried over MgSO₄. The product was further purified by preparatory HPLC (kromasil C8 column, ammonium acetate (aq, 0.1 M):acetonitrile, product came at 98% acetonitrile) to give an almost white powder (151 mg, 53% yield for 7 steps).

¹H NMR (399.964 MHz) δ 7.26-7.04 (m, 8H), 4.16-4.08 (m, 1H), 4.04-3.98 (m, 1H), 3.50-3.42 (m, 2H), 2.84-2.70 (m, 2H), 2.37 (s, 3H), 1.92 (s, 3H), 1.80-1.35 (m, 8H).

HRMS Calcd for [C₂₇H₂₉ClF₃N₃O₅S+H]⁺: 600.155. Found: 600.154.

### Example 27

### 4-[1-(2,4-dichlorophenyl)-3-({[(1S,3R)-3-hydroxycyclohexyl]amino}carbonyl)-4-methyl-1H-pyrazol-5-yl]phenyl 3,3,3-trifluoropropane-1-sulfonate and 4-[1-(2,4-dichlorophenyl-3-({[(1R,3S)-3-hydroxycyclohexyl]amino}carbonyl)-4-methyl-1H-pyrazol-5-yl]phenol 3,3,3-trifluoropropane-1-sulfonate

### Step A: 2,2,2-trichloroethyl 5-[4-(benzyloxy)phenyl]-1-(2,4-dichlorophenyl)-4-methyl-1H-pyrazole-3-carboxylate

Oxalyl chloride (20 ml) and 1 drop of DMF were added to a mixture of 5-[4-(benzyloxy)phenyl]-1-(2,4-dichlorophenyl)-4-methyl-1H-pyrazole-3-carboxylic acid, prepared as in Ex. 3, Step D (10 g, 22.06 mmol) in DCM (150 ml). The reaction was continued at room temperature for 3 hours. The solvent and excess oxalyl chloride were evaporated. The acid chloride was dissolved in DCM (100 ml) and added to a mixture of 2,2,2-trichloroethanol (4.7 g, 31.46 mmol) and DIPEA (5.0 ml, 28.70 mmol) in DCM (50 ml). DMAP (100 mg, 0.82 mmol) was added and the reaction was continued at room temperature for 2 hours. Water was added. The phases were separated and the organic phase washed with NaOH (aq), HCl (aq) and water, and dried over MgSO₄ (12.43 g, crude). ¹H NMR (399.964 MHz) δ 7.42-7.22 (m, 8H), 7.05 (d, 2H), 6.90 (d, 2H), 5.04 (s, 2H), 5.02 (s, 2H), 2.35 (s, 3H). MS m/z 583, 585, 587, 589 (M+H)⁺.

### Step B: 2,2,2-trichloroethyl 1-(2,4-dichlorophenyl)-5-(4-hydroxyphenyl)-4-methyl-1H-pyrazole-3-carboxylate

2,2,2-Trichloroethyl 5-[4-(benzyloxy)phenyl]-1-(2,4-dichlorophenyl)-4-methyl-1H-pyrazole-3-carboxylate (12.43 g, crude) was dissolved in HBr in acetic acid (33%, 110 ml) and reacted at room temperature for 2.5 hours. The mixture was cooled to 0° C., ethanol was added and the material stirred for 20 minutes. The solvent was evaporated. Methanol was added, the mixture neutralised with NaHCO₃ (5%, aq) and the solvent evaporated. Water and DCM were added. The phases separated and the organic phase washed with water and dried over MgSO₄ (9.49 g, crude). ¹H NMR (399.964 MHz) δ 7.34-7.18 (m, 3H), 6.93 (d, 2H), 6.71 (d, 2H), 6.25-6.10 (br, 1H), 5.01 (s, 2H), 2.33 (s, 3H). MS m7/z 493, 495, 497, 499 (M+H)⁺.

### Step C: 2,2,2-trichloroethyl 1-(2,4-dichlorophenyl)-4-methyl-5-(4-{[(3,33-trifluoropropyl)sulfonyl]oxy}phenyl-1H-pyrazole-3-carboxylate

3,3,3-Trifluoropropane-1-sulfonyl chloride (2.44 g, 12.42 mmol) in DCM (10 ml) was added to a mixture of 2,2,2-trichloroethyl 1-(2,4-dichlorophenyl)-5-(4-hydroxyphenyl)-4-methyl-1H-pyrazole-3-carboxylate (4.49 g, crude) and TEA (1.65 ml, 11.84 mmol) in DCM (100 ml) at −78° C., under N₂(g). The reaction was continued at −78° C. for 1 hour.

Water was added, the phases were separated and the organic phase was washed with water and dried over MgSO₄ (6.06 g, crude). ¹H NMR (399.964 MHz) δ 7.38-7.16 (m, 7H), 5.02 (s, 2H), 3.50-3.43 (m, 2H), 2.82-2.68 (m, 2H), 2.34 (s, 3H). MS m/z 653, 655, 657, 659 (M+H)⁺.

### Step D: 1-(2,4-dichlorophenyl)-4-methyl-5-(4-{[(3,3,3-trifluoropropyl)sulfonyl]oxy}phenyl)-1H-pyrazole-3-carboxylic acid

Zinc dust (6.3 g, 96.35 mmol) was added to a mixture of 2,2,2-trichloroethyl 1-(2,4-dichlorophenyl)-4-methyl-5-(4-{[(3,3,3-trifluoropropyl)sulfonyl]oxy}phenyl)-1H-pyrazole-3-carboxylate (6.06 g, crude) in acetic acid (100 ml). The reaction was continued at room temperature for 2.5 hours. DCM was added and the mixture was filtered through Celite 521. The solvent was evaporated and the mixture dissolved in DCM and washed with HCl (1M, aq) and water, and dried over MgSO₄. The mixture was dried further by coevaporation with toluene (3.75 g, crude). ¹H NMR (399.964 MHz) δ 7.76-7.64 (m, 2H), 7.58-7.50 (m, 1H), 7.40-7.28 (m, 4H), 3.90-3.82 (m, 2H), 2.95-2.80 (m, 2H), 2.21 (s, 3H). MS m/z 523, 525, 527 (M+H)⁺.

### Step E: 4-(1-(2,4-dichlorophenyl)-3-{[(3-hydroxycyclohexyl)amino]carbonyl}-4-methyl-1H-pyrazol-5-yl)phenyl 3,3,3-trifluoropropane-1-sulfonate

Oxalyl chloride (1 ml) was added to 1-(2,4-dichlorophenyl)-4-methyl-5-(4-{[(3,3,3-trifluoropropyl)sulfonyl]oxy}phenyl)-1H-pyrazole-3-carboxylic acid (314 mg, crude) in DCM (10 ml). One drop of DMF was added and the reaction continued at room temperature for 35 minutes. The solvent and excess oxalyl chloride was evaporated at reduced pressure. The acid chloride was suspended in DCM (5 ml) and added to 3-aminocyclohexanol (80 mg, 0.69 mmol) in DCM/K₂CO₃ (10%, aq) (1:1, 10 ml). The reaction was continued at room temperature for 24 hours. The phases were separated and the organic phase was washed with water and dried over MgSO₄ (389 mg, crude).

### Step F: 4-[1-(2,4-dichlorophenyl)-3-({[(1S,3R)-3-hydroxycyclohexyl]amino}carbonyl)-4-methyl-1H-pyrazol-5-yl]phenyl 3,3,3-trifluoropropane-1-sulfonate and 4-[1-(2,4-dichlorophenyl-3-({[(1R,3S)-3-hydroxycyclohexyl]amino}carbonyl)-4-methyl-1H-pyrazol-5-yl]phenyl 3,3,3-trifluoropropane-1-sulfonate

The product was separated from its isomer from Step E by prep LC (kromasil C8 column, ammonium acetate (aq, 0.1 M):acetonitrile, product came at 94% acetonitrile) to give a white powder after freeze drying (141 mg, 26% yield for 6 steps).

¹H NMR (399.964 MHz) δ 7.42-7.38 (m, 1H), 7.30-7.15 (m, 6H), 7.04 (d, 1H), 4.10-3.97 (m, 1H), 3.80-3.71 (m, 1H), 3.50-3.42 (m, 2H), 2.84-2.70 (m, 2H), 2.35 (s, 3H), 2.29-2.21 (m, 1H), 2.00-1.14 (m, 8H). HRMS Calcd for [C₂₆H₂₆Cl₂F₃N₃O₅S+H]⁺: 620.100. Found: 620.104.

### Example 28

### 4-[1-(2,4-dichlorophenyl)-3-({[(1S3S)-3-hydroxycyclohexyl]amino}carbonyl)-4-methyl-1H-pyrazol-5-yl]phenyl 3,3,3-trifluoropropane-1-sulfonate and 4-[1-(2,4-dichlorophenyl)-3-({[(1R,3R)-3-hydroxycyclohexyl]amino}carbonyl)-4-methyl-1H-pyrazol-5-yl]phenyl 3,3,3-trifluoropropane-1-sulfonate

The product was separated from its isomer, Ex. 27; Step E by prep LC (kromasil C8 column, ammonium acetate (aq, 0.1 M):acetonitrile, product came at 95% acetonitrile) to give a white powder after freeze drying (127 mg, 23% yield for 6 steps).

¹H NMR (399.964 MHz) δ 7.42-7.38 (m, 1H), 7.30-7.15 (m, 6H), 6.83 (d, 1H), 4.44-4.32 (m, 1H), 4.14-4.06 (m, 1H), 3.50-3.42 (m, 2H), 2.84-2.70 (m, 2H), 2.35 (s, 3H), 2.05-1.35 (m, 9H). HRMS Calcd for [C₂₆H₂₆Cl₂F₃N₃O₅S+H]⁺: 620.100. Found: 620.101.

### Example 29

### 1-(24-dichlorophenyl)-5-[4-(3-fluoropropoxy)phenyl]-4-methyl-N-piperidin-1-yl-1H-pyrazole-3-carboxamide

### Step A Ethyl 1-(2,4-dichlorophenyl)-5-[4-(3-fluoropropoxy)phenyl]-4-methyl-1H-pyrazole-3-carboxylate

Ethyl 1-(2,4-dichlorophenyl)-5-(4-hydroxyphenyl)-4-methyl-1H-pyrazole-3-carboxylate, prepared by debenzylation of Ex 9, Step D using a method as described in Ex. 7 Step B) (1.137 g, 2.5 mmol), 3-fluoropropan-1-ol (293 mg, 3.75 mmol) and triphenylphosphine (984 mg, 3.75 mmol) were dissolved in anhydrous THF (16 ml), then di-tert-butyl azodicarboxylate (863 mg, 3.75 mmol) was added. The resulting mixture warms and was stirred at room temperature for 3 days. Then 3-fluoropropan-1-ol (97 mg, 1.25 mmol), triphenylphosphine (327 mg, 1.25 mmol) were added, followed by di-tert-butyl azodicarboxylate (288 mg, 1.25 mmol). The resulting mixture was stirred at room temperature overnight. Trifluoroacetic acid (2 ml) was added and the resulting mixture stirred at room temperature for 2 h. EtOAc was added the organics washed with water and brine. The organic layer was dried over Na₂SO₄ and evaporated. The residue was purified by column chromatography (silica gel, hexanes/EtOAc, 10-20%). The product containing fractions were dissolved in ethanol and 3 ml of HCl (4M in dioxane) was added and the resulting mixture stirred at room temperature for 2 h. The solvents were evaporated, the residue partitioned between EtOAc and water. The organic layer was washed with water, then dried over Na₂SO₄ and evaporated. The residue was purified by column chromatography (silica gel, hexanes/EtOAc, 10-15%) to yield ethyl 1-(2,4-dichlorophenyl)-5-[4-(3-fluoropropoxy)phenyl]-4-methyl-1H-pyrazole-3-carboxylate (1.12 g, 2.23 mmol, 89%) as a yellow oil of ca. 90% purity which was used in the next transformation without further purification. ¹H NMR (400 MHz, CDCl₃) δ 7.35-7.22 (m, 3H), 7.02 (d, J=8.7 Hz, 2H), 6.80 (d, J=8.7 Hz, 2H), 4.68-4.53 (m, 2H), 4.42 (q, J=7.1 Hz, 2H), 4.07-4.03 (m, 2H), 2.30 (s, 3H), 2.18-2.08 (m, 2H), 1.40 (t, J=7.1 Hz, 3H)

### Step B 1-(2,4-dichlorophenyl)-5-[4-(3-fluoropropoxy)phenyl]-4-methyl-1H-pyrazole-3-carboxylic acid

Ethyl 1-(2,4-dichlorophenyl)-5-[4-(3-fluoropropoxy)phenyl]-4-methyl-1H-pyrazole-3-carboxylate (1.12 g, 2.23 mmol, ca. 90% pure) was dissolved in a mixture of 15 ml THF and 15 ml EtOH, then KOH (1.25 g, 22.33 mmol) dissolved in 10 ml water was added and the resulting mixture stirred at 50° C. After 3 h 30 min the reaction mixture was cooled to room temperature and the solvents were evaporated. The residue was partitioned between DCM and 1N HCl. After phase separation the aqueous layer was extracted two times with DCM. The combined organic layers were dried over MgSO₄ and evaporated to give 1-(2,4-dichlorophenyl)-5-[4-(3-fluoropropoxy)phenyl]-4-methyl-1H-pyrazole-3-carboxylic acid (1.05 g, 2.23 mmol, 99%) as a yellowish foam which was sufficiently pure for the next step. ¹H NMR (400 MHz, CDCl₃) δ 7.39-7.23 (m, 3H), 7.03 (d, J=8.7 Hz, 2H), 6.82 (d, J=8.7 Hz, 2H), 4.68-4.54 (m, 2H), 4.07-4.04 (m, 2H), 2.32 (s, 3H), 2.18-2.09 (m, 2H)

### Step C 1-(2,4-dichlorophenyl)-5-[4-(3-fluoropropoxy)phenyl]-4-methyl-1H-pyrazole-3-carbonyl chloride

1-(2,4-dichlorophenyl)-5-[4-(3-fluoropropoxy)phenyl]-4-methyl-1H-pyrazole-3-carboxylic acid (593 mg, 1.4 mmol) was dissolved in 10 ml DCM, then oxalyl chloride (200 μl, 2.36 mmol) was added, followed by 10 μl DMF. The resulting mixture was stirred for 90 min at room temperature, then the solvents were evaporated and the residue dried in oil pump vacuum to give 1-(2,4-dichlorophenyl)-5-[4-(3-fluoropropoxy)phenyl]-4-methyl-1H-pyrazole-3-carbonyl chloride (620 mg, 1.40 mmol, 99%) as a yellowish foam which was used without further purification in the next step.

### Step D General procedure for the synthesis of 1-(2,4-dichlorophenyl)-5-[4-(3 fluoropropoxy)phenyl]-4-methyl-1H-pyrazole-3-carboxamides

To a mixture of amine or amine hydrochloride (0.3 mmol) and 100 μl pyridine in 1 ml DCM was added crude 1-(2,4-dichlorophenyl)-5-[4-(3-fluoropropoxy)phenyl]-4-methyl-1H-pyrazole-3-carbonyl chloride (88 mg, 0.2 mmol) in 1 ml DCM and the resulting mixture stirred at room temperature for 2 h 30 min. The reaction mixture was washed with 2 ml of sat. NaHCO₃ and after phase separation filtered through a phase separator. The solvents were evaporated and the residue purified by preparative HPLC eluting on a reverse-phase column with 5 to 100% acetonitrile in 0.1 M NH₄Ac.

### 1-(2,4-dichlorophenyl)-5-[4-(3-fluoropropoxy)phenyl]-4-methyl-N-piperidin-1-yl-1H pyrazole-3-carboxamide

Using piperidin-1-amine hydrochloride as amine component gave 1-(2,4-dichlorophenyl)-5-[4-(3-fluoropropoxy)phenyl]-4-methyl-N-piperidin-1-yl-1H-pyrazole-3-carboxamide (36 mg, 71 μmol, 36%) as a colorless solid. ¹H NMR (500 MHz, CDCl₃) δ 7.65 (s, 1H), 7.44-7.26 (m, 3H), 7.05 (d, J=8.7 Hz, 2H), 6.84 (d, J=8.7 Hz, 2H), 4.71-4.59 (m, 2H), 4.11-4.08 (m, 2H), 2.89-2.86 (m, 4H), 2.38 (s, 3H), 2.21-2.11 (m, 2H), 1.80-1.75 (m, 4H), 1.47-1.43 (m, 2H). HRMS Calcd for [C₂₅H₂₇Cl₂FN₄O₂+H]⁺: 505.1573. Found: 505.1554.

### Example 30

### 1-(2,4-dichlorophenyl)-5-[4-(3-fluoropropoxy)phenyl]-N-[(cis)-2-hydroxycyclohexyl]-4-methyl-1H-pyrazole-3-carboxamide

Using cis-2-aminocyclohexanol hydrochloride as amine component gave 1-(2,4-dichlorophenyl)-5-[4-(3-fluoropropoxy)phenyl]-N-[(cis)-2-hydroxycyclohexyl]-4-methyl-1H-pyrazole-3-carboxamide (27 mg, 52 μmol, 26%) as a colorless solid.

¹H NMR (500 MHz, CDCl₃) δ 7.49-7.28 (m, 3H), 7.23 (d, J=8.2 Hz, 1H), 7.05 (d, J=8.7 Hz, 2H), 6.84 (d, J=8.7 Hz, 2H), 4.71-4.59 (m, 2H), 4.18-4.16 (m, 1H), 4.11-4.08 (m, 3H), 2.64-2.62 (m, 2H), 2.38 (s, 3H), 2.34 (s, 1H), 2.23-2.12 (m, 2H), 1.81-1.45 (m, 6H) HRMS Calcd for [C₂₆H₂₈Cl₂FN₃O₃+H]⁺: 520.1570. Found: 520.1558.

### Example 31

### 1-(2,4-dichlorophenyl)-N-(4,4-difluorocyclohexyl)-5-[4-(3-fluoropropoxy)phenyl]-4-methyl-1H-pyrazole-3-carboxamide

Using 4,4-difluorocyclohexylamine as amine component gave 1-(2,4-dichlorophenyl)-N-(4,4-difluorocyclohexyl)-5-[4-(3-fluoropropoxy)phenyl]-4-methyl-1H-pyrazole-3-carboxamide (48 mg, 89 μmol, 44%) as a colorless solid. ¹H NMR (500 MHz, CDCl₃) δ 7.45-7.28 (m, 3H), 7.05 (d, J=8.7 Hz, 2H), 6.88-6.84 (m, 3H), 4.71-4.59 (m, 2H), 4.11-4.08 (m, 3H), 2.38 (s, 3H), 2.25-2.10 (m, 6H), 1.98-1.86 (m, 2H), 1.71-1.64 (m, 2H).

HRMS Calcd for [C₂₆H₂₆Cl₂F₃N₃O₂+H]⁺: 540.1432. Found: 540.1447.

### Example 32

### 1-(2,4-dichlorophenyl)-5-[4-(3-fluoropropoxy)phenyl]-4-methyl-N-(5-methylpyridin-2-yl)-1H-pyrazole-3-carboxamide

Using 2-amino-5-picoline as amine component gave 1-(2,4-dichlorophenyl)-5-[4-(3-fluoropropoxy)phenyl]-4-methyl-N-(5-methylpyridin-2-yl)-1H-pyrazole-3-carboxamide (48 mg, 93 μmol, 47%) as a colorless solid. ¹H NMR (500 MHz, CDCl₃) δ 9.40 (s, 1H), 8.28 (d, J=8.3 Hz, 1H), 8.14 (d, J=2.2 Hz, 1H), 7.57-7.55 (m, 1H), 7.45-7.28 (m, 3H), 7.08 (d, J=8.7 Hz, 2H), 6.86 (d, J=8.7 Hz, 2H), 4.71-4.60 (m, 2H), 4.12-4.09 (m, 2H), 2.44 (s, 3H), 2.32 (s, 3H), 2.23-2.13 (m, 2H). HRMS Calcd for [C₂₆H₂₃Cl₂FN₄O₂+H]⁺: 513.1260. Found: 513.1245.

The following compounds are prepared in a similar manner to those described above:

### Example 33

1-(2,4-Dichlorophenyl)-4-methyl-5-[4-(3,3,3-trifluoropropoxy)-phenyl]-1H-pyrazole-3-carboxylic acid (2-hydroxycyclohexyl)amide

### Example 34

1-(2,4-Dichlorophenyl)-4-methyl-5-[4-(3,3,3-trifluoro-propoxy)-phenyl]-1H-pyrazole-3-carboxylic acid (3-hydroxy-cyclohexyl)amide

### Example 35

3-Fluoropropane-1-sulfonic acid 4-[2-(2,4-dichlorophenyl)-5-((1S,2R)-2-hydroxycyclohexylcarbamoyl)-4-methyl-2H-pyrazol-3-yl]phenyl ester

### Example 36

4,4,4-Trifluorobutane-1-sulfonic acid 4-[2-(3-cyano-5-fluorophenyl)-5-(1-ethylbutylcarbamoyl)-4-methyl-2H-pyrazol-3-yl]phenyl ester

### Example 37

3,3,3-Trifluoropropane-1-sulfonic acid 4-[2-(3-cyano-5-fluorophenyl)-5-(4,4-difluorocyclohexylcarbamoyl)-4-methyl-2H-pyrazol-3-yl]phenyl ester

### Example 38

3,3,3-Trifluoropropane-1-sulfonic acid 4-[2-(3-cyanophenyl)-5-(4,4-difluoro-cyclohexylcarbamoyl)-4-methyl-2H-pyrazol-3-yl]phenyl ester

### Example 39

3,3,3-Trifluoropropane-1-sulfonic acid 4-[5-(2-aminocyclohexylcarbamoyl)-2-(3-cyano-5-fluorophenyl)-4-methyl-2H-pyrazol-3-yl]-phenyl ester

### Example 40

3,3,3-Trifluoropropane-1-sulfonic acid 4-[2-(3-cyano-5-fluorophenyl) -5-(3-dimethylamino-cyclohexylcarbamoyl)-4-methyl-2H-pyrazol-3-yl]-phenyl ester

### Example 41

3,3,3-Trifluoropropane-1-sulfonic acid 4-[2-(3-cyano-5-fluorophenyl)-5-((1S,2R)-2-hydroxy-cyclohexylcarbamoyl)-4-methyl-2H-pyrazol-3-yl]phenyl ester

### Example 42

3,3,3-Trifluoropropane-1-sulfonic acid 4-[2-(3-cyanophenyl)-5-(2-hydroxy-cyclohexylcarbamoyl)-4-methyl-2H-pyrazol-3-yl]-phenyl ester

### Example 43

3,3,3-Trifluoropropane-1-sulfonic acid 4-[2-(3-cyano-5-fluoro-phenyl)-5-(3-hydroxy-cyclohexylcarbamoyl)-4-methyl-2H-pyrazol-3-yl]-phenyl ester

### Example 44

### N-cyclohexyl-1-(2,4-dichlorophenyl)-5-[4-(3-fluoropropoxy)phenyl]-4-methyl-1H-pyrazole-3-carboxamide

Using cyclohexylamine as amine component and reacting with the product of Ex. 29 Step C gave N-cyclohexyl-1-(2,4-dichlorophenyl)-5-[4-(3-fluoropropoxy)phenyl]-4-methyl-1H-pyrazole-3-carboxamide (39 mg, 77 μmol, 39%) as a colorless solid. ¹H NMR (500 MHz, CDCl₃) δ 7.44-7.28 (m, 3H), 7.05 (d, J=8.7 Hz, 2H), 6.86-6.83 (m, 3H), 4.71-4.59 (m, 2H), 4.11-4.08 (m, 2H), 4.01-3.93 (m, 1H), 2.38 (s, 3H), 2.22-2.12 (m, 2H), 2.06-2.02 (m, 2H), 1.79-1.75 (m, 2H), 1.67-1.64 (m, 1H), 1.47-1.38 (m, 2H), 1.31-1.16 (m, 3H). HRMS Calcd for [C₂₆H₂₈Cl₂FN₃O₂+H]⁺: 504.1621. Found: 504.1630.

### Example 45

3,3,3-Trifluoropropane-1-sulfonic acid 4-[2-(2-chlorophenyl)-5-(2-hydroxy-cyclohexylcarbamoyl)-4-methyl-2H-pyrazol-3-yl]phenyl ester

### Example 46

3,3,3-Trifluoropropane-1-sulfonic acid 4-[2-(2-chlorophenyl)-5-(4,4-difluorocyclohexylcarbamoyl)-4-methyl-2H-pyrazol-3-yl]phenyl ester.

1. A compound of formula (I) 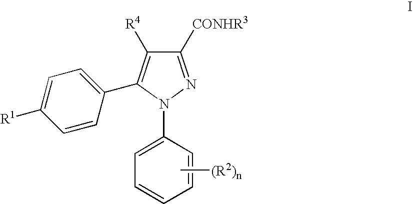
wherein
R¹ represents a group R⁵⁰— in which R⁵ represents a C₃₋₇alkyl group substituted by one or more fluoro or R⁵ represents a C₃₋₇alkylsulphonyl group which is optionally substituted by one or more fluoro;
R² represents a C₁₋₄alkyl group, hydroxy, fluoro, chloro or cyano wherein each R² is independently selected when n is >1;
R³ represents a) cyclohexyl optionally substituted by one or more of the following: hydroxy, fluoro, amino, mono or diC₁₋₃alkylamino, carboxy or a C₁₋₄alkoxycarbonyl group b) piperidino substituted by one or more hydroxy c) unsubstituted piperidino but only when one of the following applies: R⁴ represents cyano or R¹ represents 3-fluoropropylsulphonyloxy or R¹ represents 3,3,3-trifluoropropoxy or R¹ represents 3-fluoropropoxy or R² is methyl d) phenyl substituted by one or more of the following: hydroxy, halo or a C₁₋₄alkyl group e) pyridyl substituted by a C₁₋₄alkyl group or f) a C₄₋₉alkyl group;
R⁴ represents cyano or methyl; and
n is 1, 2 or 3;
or a pharmaceutically acceptable salt thereof.

2. The compound according to claim 1 wherein R¹ represents n-butylsulfonyloxy, n-propylsulfonyloxy, 3-methylbutylsulfonyloxy, 4,4,4-trifluorobutyl-1-sulfonyloxy, 4-fluorobutyl-1-sulfonyloxy, 3,3,3-trifluoropropyl-1-sulfonyloxy, 3-fluoropropyl-1-sulfonyloxy, 4,4,4-trifluorobutoxy, 4-fluorobutoxy, 3,3,3-trifluoropropoxy or 3-fluoropropoxy.

3. The compound according to claim 1 wherein R² represents chloro, fluoro, cyano, hydroxy or methyl and n is 1, 2 or 3.

4. The compound according to claim 1 wherein R³ represents cyclohexyl substituted by one or more of the following: hydroxy, fluoro, amino, mono or di C₁₋₃alkylamino, carboxy or a C₁₋₄alkoxycarbonyl group;

5. The compound according to claim 1 wherein R³ represents piperidino substituted by one or more hydroxy.

6. The compound according to claim 1 wherein R³ represents unsubstituted piperidino but only when one of the following applies: R⁴ represents cyano or R¹ represents 3-fluoropropylsulphonyloxy or R¹ represents 3,3,3-trifluoropropoxy or R¹ represents 3-fluoropropoxy or R² is methyl.

7. The compound according to claim 1 wherein R³ represents phenyl substituted by one or more of the following: hydroxy, halo or a C₁₋₄alkyl group.

8. The compound according to claim 1 wherein R³ represents pyridyl substituted by a C₁₋₄alkyl group or fluoro.

9. The compound according to claim 1 wherein R³ represents a C₄₋₉alkyl group.

10. The compound according to claim 1 wherein R⁴ represents cyano.

11. The compound according to claim 1 wherein R⁴ represents methyl.

12. The compound according to claim 1 selected from:
4-{4-cyano-1-(2,4-dichlorophenyl)-3-[(piperidin-1-ylamino)carbonyl]-1H-pyrazol-5-yl}phenyl 3,3,3-trifluoropropane-1-sulfonate;
4-{4-cyano-1-(2,4-dichlorophenyl)-3-[(piperidin-1-ylamino)carbonyl]-1H-pyrazol-5-yl}phenyl 3-methylbutane-1-sulfonate;
4-[1-(2,4-dichlorophenyl)-3-({[(1R,2S)-2-hydroxycyclohexyl]amino}carbonyl)-4-methyl-1H-pyrazol-5-yl]phenyl 3,3,3-trifluoropropane-1-sulfonate;
4-[1-(2,4-dichlorophenyl)-3-({[(1S,2R)-2-hydroxycyclohexyl]amino}carbonyl)-4-methyl-1H-pyrazol-5-yl]phenyl 3,3,3-trifluoropropane-1-sulfonate;
4-(1-(2,4-dichlorophenyl)-3-{[(5-fluoropyridin-2-yl)amino]carbonyl}-4-methyl-1H-pyrazol-5-yl)phenyl 3,3,3-trifluoropropane-1-sulfonate;
4-(1-(2,4-dichlorophenyl)-3-{[(3,4-difluoro-2-hydroxyphenyl)amino]carbonyl}-4-methyl-1H-pyrazol-5-yl)phenyl-3,3,3-trifluoropropane-1-sulfonate;
4-{1-(2,4-dichlorophenyl)-4-methyl-3-[(piperidin-1-ylamino)carbonyl]-1H-pyrazol-5-yl}phenyl 3-fluoropropane-1-sulfonate;
3,3,3-trifluoropropane-1-sulfonic acid 4-[2-(2,4-dichlorophenyl)-5-(4-hydroxy-piperidin-1-ylcarbamoyl)-4-methyl-2H-pyrazol-3-yl]phenyl ester;
3,3,3-trifluoropropane-1-sulfonic acid 4-[2-(2,4-dichlorophenyl)-5-(3-hydroxy-piperidin-1-ylcarbamoyl)-4-methyl-2H-pyrazol-3-yl]phenyl ester;
3-methylbutane-1-sulfonic acid 4-[2-(2,4-dichlorophenyl)-4-methyl-5-(5-methyl-pyridin-2-ylcarbamoyl)-2H-pyrazol-3-yl]phenyl ester;
3,3,3-trifluoropropane-1-sulfonic acid 4-[2-(2,4-dichlorophenyl)-4-methyl-5-(5-methyl-pyridin-2-ylcarbamoyl)-2H-pyrazol-3-yl]phenyl ester;
(−)-4-[1-(2,4-dichlorophenyl)-3-({[cis-2-hydroxycyclohexyl]amino}carbonyl)-4-methyl-1H-pyrazol-5-yl]phenyl 3,3,3-trifluoropropane-1-sulfonate;
(+)-4-[1-(2,4-dichlorophenyl)-3-({[cis-2-hydroxycyclohexyl]amino}carbonyl)-4-methyl-1H-pyrazol-5-yl]phenyl 3,3,3-trifluoropropane-1-sulfonate;
4-[1-(2,4-dichlorophenyl)-3-({[3-(dimethylamino)cyclohexyl]amino}carbonyl)-4-methyl-1H-pyrazol-5-yl]phenyl 3,3,3-trifluoropropane-1-sulfonate;
4-[1-(2,4-dichlorophenyl)-3-({[trans-3-(dimethylamino)cyclohexyl]amino}carbonyl)-4-methyl-1H-pyrazol-5-yl]phenyl 3,3,3-trifluoropropane-1-sulfonate;
4-[1-(2,4-dichlorophenyl)-3-({[cis-3-(dimethylamino)cyclohexyl]amino}carbonyl)-4-methyl-1H-pyrazol-5-yl]phenyl 3,3,3-trifluoropropane-1-sulfonate;
4-[3-({[cis-3-aminocyclohexyl]amino}carbonyl)-1-(2,4-dichlorophenyl)-4-methyl-1H-pyrazol-5-yl]phenyl 3,3,3-trifluoropropane-1-sulfonate;
4-[3-[({trans-3-[(tert-butoxycarbonyl)amino]cyclohexyl}amino)carbonyl]-1-(2,4-dichlorophenyl)-4-methyl-1H-pyrazol-5-yl]phenyl 3,3,3-trifluoropropane-1-sulfonate;
1-(2,4-dichlorophenyl)-4-methyl-N-piperidin-1-yl-5-[4-(3,3,3-trifluoropropoxy)phenyl]-1H-pyrazole-3-carboxamide;
N-cyclohexyl-1-(2,4-dichlorophenyl)-4-methyl-5-[4-(3,3,3-trifluoropropoxy)phenyl]-1H-pyrazole-3-carboxamide;
1-(2,4-dichlorophenyl)-N-[(cis)-2-hydroxycyclohexyl]-4-methyl-5-[4-(3,3,3-trifluoropropoxy)phenyl]-1H-pyrazole-3-carboxamide;
1-(2,4-dichlorophenyl)-N-(4,4-difluorocyclohexyl)-4-methyl-5-[4-(3,3,3-trifluoropropoxy)phenyl]-1H-pyrazole-3-carboxamide;
1-(2,4-dichlorophenyl)-4-methyl-N-(5-methylpyridin-2-yl)-5-[4-(3,3,3-trifluoropropoxy)phenyl]-1H-pyrazole-3-carboxamide;
4-[1-(2-chlorophenyl)-3-{[(1S,2R)-2-hydroxycyclohexyl]carbamoyl}-4-methyl-1H-pyrazol-5-yl]phenyl 3,3,3-trifluoropropane-1-sulfonate;
4-[1-(2-chlorophenyl)-3-{[(1R,2S)-2-hydroxycyclohexyl]carbamoyl}-4-methyl-1H-pyrazol-5-yl]phenyl 3,3,3-trifluoropropane-1-sulfonate:
4-[1-(2-chlorophenyl)-3-(cyclohexylcarbamoyl)-4-methyl-1H-pyrazol-5-yl]phenyl 3,3,3-trifluoropropane-1-sulfonate;
4-{1-(4-chloro-2-methylphenyl)-4-methyl-3-[(piperidin-1-ylamino)carbonyl]-1H-pyrazol-5-yl}phenyl 3,3,3-trifluoropropane-1-sulfonate;
4-[1-(4-chloro-2-methylphenyl)-3-({[(1S,2R)-2-hydroxycyclohexyl]amino}carbonyl)-4-methyl-1H-pyrazol-5-yl]phenyl 3,3,3-trifluoropropane-1-sulfonate;
4-[1-(4-chloro-2-methylphenyl)-3-({[(1R,2S)-2-hydroxycyclohexyl]amino}carbonyl)-4-methyl-1H-pyrazol-5-yl]phenyl 3,3,3-trifluoropropane-1-sulfonate;
4-[1-(2,4-dichlorophenyl)-3-({[(1S,3R)-3-hydroxycyclohexyl]amino}carbonyl)-4-methyl-1H-pyrazol-5-yl]phenyl 3,3,3-trifluoropropane-1-sulfonate;
4-[1-(2,4-dichlorophenyl)-3-({[(1R,3S)-3-hydroxycyclohexyl]amino}carbonyl)-4-methyl-1H-pyrazol-5-yl]phenyl 3,3,3-trifluoropropane-1-sulfonate;
4-[1-(2,4-dichlorophenyl)-3-({[(1S,3S)-3-hydroxycyclohexyl]amino}carbonyl)-4-methyl-1H-pyrazol-5-yl]phenyl 3,3,3-trifluoropropane-1-sulfonate;
4-[1-(2,4-dichlorophenyl)-3-({[(1R,3R)-3-hydroxycyclohexyl]amino}carbonyl)-4-methyl-1H-pyrazol-5-yl]phenyl 3,3,3-trifluoropropane-1-sulfonate;
1-(2,4-dichlorophenyl)-5-[4-(3-fluoropropoxy)phenyl]-4-methyl-N-piperidin-1-yl-1H-pyrazole-3-carboxamide;
1-(2,4-dichlorophenyl)-5-[4-(3-fluoropropoxy)phenyl]-N-[(cis)-2-hydroxycyclohexyl]-4-methyl-1H-pyrazole-3-carboxamide;
1-(2,4-dichlorophenyl)-N-(4,4-difluorocyclohexyl)-5-[4-(3-fluoropropoxy)phenyl]-4-methyl-1H-pyrazole-3-carboxamide;
1-(2,4-dichlorophenyl)-5-[4-(3-fluoropropoxy)phenyl]-4-methyl-N-(5-methylpyridin-2-yl)-1H-pyrazole-3-carboxamide;
1-(2,4-Dichlorophenyl)-4-methyl-5-[4-(3,3,3-trifluoropropoxy)-phenyl]-1H-pyrazole-3-carboxylic acid (2-hydroxycyclohexyl)amide 1-(2,4-dichlorophenyl)-4-methyl-5-[4-(3,3,3-trifluoropropoxy)phenyl]-1H-pyrazole-3-carboxylic acid (3-hydroxycyclohexyl)amide;
3-fluoropropane-1-sulfonic acid 4-[2-(2,4-dichlorophenyl)-5-((1S,2R)-2-hydroxycyclohexylcarbamoyl)-4-methyl-2H-pyrazol-3-yl]phenyl ester;
4,4,4-trifluorobutane-1-sulfonic acid 4-[2-(3-cyano-5-fluorophenyl)-5-(1-ethylbutylcarbamoyl)-4-methyl-2H-pyrazol-3-yl]phenyl ester;
3,3,3-trifluoropropane-1-sulfonic acid 4-[2-(3-cyano-5-fluorophenyl)-5-(4,4-difluoro-cyclohexylcarbamoyl)-4-methyl-2H-pyrazol-3-yl]phenyl ester;
3,3,3-trifluoropropane-1-sulfonic acid 4-[2-(3-cyanophenyl)-5-(4,4-difluoro-cyclohexylcarbamoyl)-4-methyl-2H-pyrazol-3-yl]phenyl ester;
3,3,3-trifluoropropane-1-sulfonic acid 4-[5-(2-aminocyclohexylcarbamoyl)-2-(3-cyano-5-fluorophenyl)-4-methyl-2H-pyrazol-3-yl]-phenyl ester;
3,3,3-trifluoropropane-1-sulfonic acid 4-[2-(3-cyano-5-fluorophenyl) -5-(3-dimethylaminocyclohexylcarbamoyl)-4-methyl-2H-pyrazol-3-yl]phenyl ester;
3,3,3-trifluoropropane-1-sulfonic acid 4-[2-(3-cyano-5-fluorophenyl)-5-((1S,2R)-2-hydroxycyclohexylcarbamoyl)-4-methyl-2H-pyrazol-3-yl]phenyl ester;
3,3,3-trifluoropropane-1-sulfonic acid 4-[2-(3-cyanophenyl)-5-(2-hydroxy-cyclohexylcarbamoyl)-4-methyl-2H-pyrazol-3-yl]phenyl ester;
3,3,3-trifluoropropane-1-sulfonic acid 4-[2-(3-cyano-5-fluorophenyl)-5-(3-hydroxy-cyclohexylcarbamoyl)-4-methyl-2H-pyrazol-3-yl]phenyl ester;
N-cyclohexyl-1-(2,4-dichlorophenyl)-5-[4-(3-fluoropropoxy)phenyl]-4-methyl-1H-pyrazole-3-carboxamide;
3,3,3-trifluoropropane-1-sulfonic acid 4-[2-(2-chlorophenyl)-5-(2-hydroxy-cyclohexylcarbamoyl)-4-methyl-2H-pyrazol-3-yl]phenyl ester; and
3,3,3-trifluoropropane-1-sulfonic acid 4-[2-(2-chlorophenyl)-5-(4,4-difluorocyclohexylcarbamoyl)-4-methyl-2H-pyrazol-3-yl]phenyl ester;
and pharmaceutically acceptable salts thereof.

13. (canceled)

14. A pharmaceutical formulation comprising a compound of formula I as claimed in claim 1 or claim 12 and a pharmaceutically acceptable adjuvant, diluent or carrier.

15. (canceled)

16. A method of treating obesity, psychiatric disorders, psychotic disorders, schizophrenia and bipolar disorders, anxiety, anxio-depressive disorders, depression, cognitive disorders, memory disorders, obsessive-compulsive disorders, anorexia, bulimia, attention disorders, epilepsy and related conditions, neurological disorders, Parkinson's Disease, Huntington's Chorea and Alzheimer's Disease, immune, cardiovascular, reproductive and endocrine disorders, septic shock, diseases related to the respiratory and gastrointestinal system, and extended abuse, addiction and/or relapse indications, comprising administering a pharmacologically effective amount of a compound of formula I as claimed in as claimed claim 1 to a patient in need thereof.

17. (canceled)

18. A process for the preparation of a compound of formula I as claimed in claim 1 comprising
a) reacting a compound of formula II 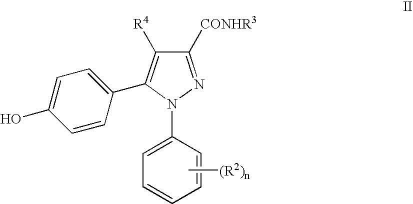
in which R², R³, R⁴, and n are as previously defined with a group R^{1A}-X in which R^{1A} represents a group such that R^{1A}O represents R¹ and X represents a leaving group at a temperature in the range of −25 to 150° C., in the presence of an inert solvent and optionally in the presence of a base to give a compound of formula I in which R¹ represents a) a C₃₋₆alkoxy group substituted by one or more fluoro or b) a group of formula phenyl(CH₂)ₚO— in which p is 1, 2 or 3 and the phenyl ring is optionally substituted by 1, 2 or 3 groups represented by Z, or c) a group R⁵S(O)₂O; or
b) reacting a compound of formula III 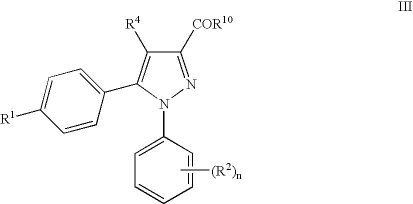
in which R¹, R², R⁴ and n are as previously defined and R¹⁰ represents OH or a C₁₋₆alkoxy group or chloro with a compound of formula IV or a salt thereof
R³NH₂ IV
in which R³ is as previously defined, in an inert solvent in the presence of a Lewis Acid at a temperature in the range of −25° C. to 150° C. when R¹⁰ is a C₁₋₆alkoxy group; or alternatively when R¹⁰ is OH by reacting a compound of formula III with a chlorinating agent and then reacting the acid chloride produced with an amine of formula IV in an inert solvent in the presence of a base at a temperature in the range of −25° C. to 150° C. to give a compound of formula I in which R¹, R², R³, R⁴, and n are as previously defined.
